(19) United States
(12) Patent Application Publication
(10) Pub. No.: US 2016/0115542 A1
(43) Pub. Date: Apr. 28, 2016

(54) BIOMARKER OF Nrf2 ACTIVATION

(71) Applicants: MOCHIDA PHARMACEUTICAL CO., LTD., Tokyo (JP); TOHOKU UNIVERSITY, Miyagi (JP)

(72) Inventors: Masayuki Yamamoto, Miyagi (JP); Akira Uruno, Miyagi (JP); Yuki Furusawa, Tokyo (JP)

(73) Assignees: Mochida Pharmaceutical Co., Ltd., Shinjuku-ku, Tokyo (JP); Tohoku University, Aoba-ku, Sendai-shi, Miyagi (JP)

(21) Appl. No.: 14/894,031

(22) PCT Filed: May 28, 2014
(86) PCT No.: PCT/JP2014/064074

§ 371 Date: Nov. 25, 2015

(30) Foreign Application Priority Data
May 29, 2013 (JP) 2013-112877

(51) Int. Cl. C12Q 1/68; G01N 33/68

(52) U.S. Cl. CPC C12Q 1/6883; G01N 33/6893; G01N 2333/50; C12Q 2600/158; C12Q 2600/106; G01N 2500/04; G01N 2800/52; G01N 2500/10

(57) ABSTRACT

Provided is FGF21 that is a metabolism-related biomarker reflecting the activation of Nrf2 in vivo. Also provided are a method for monitoring a response of a subject to which an Nrf2 activator is administered, said method comprising a step for measuring the FGF21 level, at a point during or after the administration of the Nrf2 activator, in a biosample that is derived from the subject, wherein an increase in the FGF21 level indicates a positive response to the administration of the Nrf2 activator, etc. According to the present invention, a metabolism-related biomarker that reflects the activation of Nrf2 in vivo is provided.

### TECHNICAL FIELD

The present invention relates to a metabolism-related biomarker of Nrf2 activation, and use thereof.

### BACKGROUND ART

There are increasing demands for more effective therapies based on novel mechanism against metabolic disorders such as diabetes mellitus, hyperlipidemia and obesity.

Recently, FGF 21 (fibroblast growth factor 21) has attracted interest as a development target for new therapeutic agents for metabolic disorders. FGF21 is identified as one of the FGF family molecules (PTL 1, NPL 1). It has been found that FGF21 regulates glucose and lipid metabolism, and a method is reported that includes administering FGF21 for the treatment of diabetes mellitus and obesity (PTL 2). There are reports that administration of FGF21 improves hyperglycemia, insulin resistance and hyperlipidemia and attenuates weight gain in diabetic model animals. There is also development of an FGF21-related substance as a therapeutic agent for metabolic disorders such as diabetes mellitus.

There are reports to directly inject FGF21 protein or introduce FGF21 gene as a method of increasing blood FGF21 level, and most of the substances that are currently developed are derivatives or modification of FGF21 protein. PPARα and PPARγ activation is reported as a mechanism that indirectly promotes FGF21 production. NPL 2 and NPL3 describe PPARα and PPARβ activation and other such mechanisms promoting FGF21 production. PTL 3 describes a PPARα activator as a compound having an FGF21 production promoting effect.

FGF21 is expressed in liver, white adipose tissue and pancreas, and secreted into plasma. PTL 4 describes using FGF21 as a sirtuin biomarker.

Nrf2 (nuclear factor E2-related factor 2) is a basic region-leucine zipper-type transcription factor belonging to the Cap ‘n’ collar (CNC) family. Nrf2 plays important roles in the protection against oxidative stress, and cooperatively induces cytoprotective genes. Nrf2 is negatively regulated by Keap1 (Kelch-like ECH-associated protein 1) through ubiqutination and proteasome-mediated degradation under unstressed condition. When Keap1 is exposed to oxidative and electrophilic stimuli, Nrf2 is avoided from the proteasome-mediated degradation. Thereafter Nrf2 translocates and accumulates in the nucleus and forms heterodimer with small Maf proteins (sMaf), and binds to the antioxidant responsive element (ARE) in the antioxidant and detoxification genes and induces their expression. This stress-response gene regulation system is called ‘the Keap1-Nrf2 system’.

Although the Keap1-Nrf2 system plays pivotal roles in antioxidant and detoxification functions, the Keap1-Nrf2 system is also involved in metabolic homeostasis regulation. Nrf2 knockout (Nrf2^{-/-}) mice are resistant to weight gain in high-fat-diet (HFD)-induced obesity model and show ameliorated insulin resistance (NPL 4). Meanwhile, Nrf2 deficiency impaired glucose tolerance and aggravated hyperglycemia in streptozotocin (STZ)-induced diabetic model mice and ob/ob obese model mice (NPL 5 and NPL6). Nrf2 is also involved in glucose and glutamine metabolism in cancer cells.

Effects of Nrf2 activators against metabolic disorders also have been reported. There is also a report that sulforaphane, a compound with potential to activate Nrf2, protected β cell injury in STZ-induced type 1 diabetic mice (NPL 7). It has also been reported that lithospermic acid B (LAB), a compound with potential to activate Nrf2, protected β cell injury in type 2 diabetes model rats (NPL 8). There is also a report that oltipraz, a schistosomicide that is currently under clinical trials as an anticancer drug, is a potent Nrf2 activator, and attenuates HFD-induced insulin resistance and obesity (NPL 9). CDDO-Me (2-cyano-3,12-dioxooleana-1,9(11)-dien-28-oic acid methyl ester), which had been under clinical trials as a therapeutic agent for diabetic nephropathy in the United States, is described as a potent Nrf2 activator (NPL 10), and has been shown to reduce blood glucose level and plasma lipid level in HFD-induced obesity model and diabetic db/db model mice (NPL 11). It has also been reported that repetitive administration of a CDDO-Me derivative CDDO-Im (1-[2-cyano-3,12-dioxooleana-1,9(11)-dien-28-oyl]imidazole) prevents high-fat-diet (HFD)-induced obesity by increasing energy expenditure (NPL 12).

These studies show significance of Nrf2 as a regulatory factor of metabolic homeostasis, as well as detoxification and cytoprotection, and propose that Nrf2 is a novel candidate pharmacological target for improving metabolic disorders such as diabetes mellitus.

Concerning the link between Nrf2 and FGF21, there are reports that Nrf2 negatively regulates FGF21 expression using Nrf2 knockout mice and Keap1 knockdown mice (NPL 13 to 16). However, there is no report of Nrf2 positively regulating FGF21 expression.

As described above, the precise mechanism of metabolic regulation by Nrf2 has not been fully elucidated, and metabolism-related Nrf2 target genes have not been identified.

### CITATION LIST

### Patent Literature

PTL 1: WO01/036640
PTL 2: WO03/011213
PTL 3: WO2011/037223
PTL 4: WO2008/115518

### Non Patent Literature

NPL 1: Nishimura et al. (2000) Biochimica et Biophyisica Acta, 1492, 203-206
NPL 2: Galman et al. (2008) Cell Metabolism, 8, 169-174
NPL 3: Muise et al. (2008) Molecular Pharmacology, 74, 403-412
NPL 4: Meher et al. (2012) Free Radical Biology and Medicine, 52, 1708-1715
NPL 5: Aleksunes et al. (2010) The Journal of Pharmacology and Experimental Therapeutics, 333, 140-151
NPL 6: Xue et al. (2013) Diabetes, 62, 845-854
NPL 7: Song et al. (2009) Toxicology and Applied Pharmacology, 235, 57-67
NPL 8: Lee et al. (2011) Toxicology and Applied Pharmacology, 252, 47-54
NPL 9: Yu et al. (2011) Diabetologia, 54, 922-934
NPL 10: Pergola et al. (2011) The New England Journal of Medicine, 365, 327-336
NPL 11: Saha et al. (2010) The Journal of Biological Chemistry, 285, 40581-40592
NPL 12: Shin et al. (2009) European Journal of Pharmacology, 620, 138-144
NPL 13: Chartoumpekis et al. (2011) Diabetes, 60, 2465-2473
NPL 14: Zhang et al. (2010) Toxicology and Applied Pharmacology, 245, 326-334
NPL 15: Lu et al. (2011) Toxicology and Applied Pharmacology, 256, 122-135
NPL 16: Zhang et al. (2012) Toxicology and Applied Pharmacology, 264, 305-314

### SUMMARY OF INVENTION

### Technical Problem

In drug development process, biomarkers are required to predict efficacy and safety of novel candidate of drugs. However, metabolism-related target genes remain to be identified in Nrf2 activation, and metabolism-related markers directly involved in improving metabolic disorders, and that are measurable in blood or urine samples in a convenient and low invasive fashion are not known to date. There accordingly is a need for a metabolism-related biomarker that reflects Nrf2 activation in vivo for the development of Nrf2 activators.

### Solution to Problem

The present inventors have shown roles of Nrf2 activation in diabetic model mice, and characterized its involvement in FGF21 regulation. Nrf2 activation by knockdown of Keap1 gene increased plasma FGF21 expression level and hepatic Fgf21 gene expression level in diabetic db/db mice and high-calorie-induced obesity model mice. Administration of an Nrf2 activator CDDO-Im also increased plasma FGF21 expression level and hepatic Fgf21 gene expression level in db/db mice, but not in Nrf2 gene knockout db/db mice.

The present inventors also found that Nrf2 positively regulates FGF21 in diabetic mice, and that FGF21 is a biomarker that mediates metabolic regulations by the Keap1-Nrf2 system. The present invention was completed on the basis of these findings.

Specifically, the present invention provides, for example, a method for monitoring a response of a subject administered with an Nrf2 activator, a method for monitoring a response of a subject treated with an Nrf2 activator, a method for predicting a response to a treatment with an Nrf2 activator, a method for selecting the optimum dose of an Nrf2 activator, and a method for screening for a preventive or therapeutic agent for diabetes mellitus, as follows.

[1] A method for monitoring a response of a subject administered with an Nrf2 activator,

the method comprising the step of measuring FGF21 level in a biological sample derived from the subject at a time point during or after the administration of the Nrf2 activator,

wherein increase of FGF21 level indicates a positive response to the administration of the Nrf2 activator.

[2] A method for monitoring a response of a subject treated with an Nrf2 activator,

the method comprising the step of measuring FGF21 level in a biological sample derived from the subject at a time point during or after the administration of the Nrf2 activator,

wherein increase of FGF21 level indicates a positive response to the treatment with the Nrf2 activator.

[3] A method for predicting a clinical response to a treatment with an Nrf2 activator in a subject,

the method comprising the step of measuring FGF21 level in a biological sample derived from the subject at a time point during or after the administration of the Nrf2 activator,

wherein increase of FGF21 level indicates high sensitivity and/or high reactivity to the treatment with the Nrf2 activator.

[4] A method for selecting a subject for which a treatment with the Nrf2 activator is effective,

the method comprising the step of measuring FGF21 level in a biological sample derived from the subject at a time point during or after the administration of the Nrf2 activator,

wherein increase of FGF21 level indicates that the treatment with the Nrf2 activator is effective for the subject.

[5] A method for selecting the optimum dose of an Nrf2 activator for the treatment of a subject with the Nrf2 activator,

the method comprising the step of measuring FGF21 level in a biological sample derived from the subject at a time point during or after the administration of the Nrf2 activator,

wherein the optimum dose of the Nrf2 activator is determined by using increase of FGF21 level as an index.

[6] The method according to [1] or [2], wherein the subject is a subject that has had continuous administration of the Nrf2 activator.

[7] The method according to [1], wherein the subject is a healthy subject.

[8] The method according to anyone of [1] to [6], wherein the subject is a subject affected by at least one disorder selected from the group consisting of diabetes mellitus, obesity, hypertriglyceridemia, ischemic cerebrovascular disease, ischemic cardiovascular disease, and diabetic nephropathy.

[9] The method according to anyone of [1] to [8], wherein the increase of FGF21 level is increase of FGF21 level relative to an FGF21 level selected from the group consisting of the following (a) to (d):

(a) an FGF21 level in a biological sample derived from the subject at a time point before the administration of the Nrf2 activator;

(b) an FGF21 level in a biological sample derived from a reference population;

(c) a predetermined FGF21 level; and

(d) an FGF21 level in a biological sample derived from the subject at a time point before the measurement of FGF21 level.

[10] The method according to any one of [1] to [9], wherein the FGF21 level is FGF21 protein level or FGF21 mRNA level.

[11] The method according to [10], wherein the FGF21 level is FGF21 protein level.

[12] The method according to [10] or [11], wherein the measurement is performed by immunoassay.

[13] The method according to any one of [1] to [12], wherein the biological sample is a body fluid.

[14] The method according to [13], wherein the body fluid is selected from the group consisting of whole blood, serum, and plasma.

[15] A method for screening for a preventive or therapeutic agent for Nrf2- and/or FGF21-related disorder using Nrf2 activation activity as an index,

the method comprising the steps of:

1) comparing Nrf2 activity in (i) an Nrf2 expressing cell contacted to a test compound, and (ii) an Nrf2 expressing cell not contacted to the test compound; and

2) selecting a test compound that increases the Nrf2 activity in (i) relative to the Nrf2 activity in (ii), the Nrf2 activity being measured by using increase of FGF21 expression level as an index.

[16] The method according to [15], wherein the Nrf2 activity is measured by using increase of FGF21 expression level in a hepatocyte as an index.

[17] A method for screening for a preventive or therapeutic agent for Nrf2- and/or FGF21-related disorder using Nrf2 activation activity as an index, the method comprising the steps of:

1) comparing Nrf2 activity in (i) an Nrf2 expressing cell contacted to a test compound, and (ii) an Nrf2 expressing cell not contacted to the test compound;

2) selecting a test compound that increases the Nrf2 activity in (i) relative to the Nrf2 activity in (ii); and

3) checking that the test compound selected in the step 2) has activity that increases FGF21 expression level.

[18] The method according to [17], wherein, in the step 1), the Nrf2 activity is measured by using Nrf2 stabilization as an index.

[19] The method according to [17] or [18], wherein the step 3) checks for activity that increases FGF21 expression level in a hepatocyte.

[20] The method according to any one of [15] to [19], wherein the disorder is at least one selected from the group consisting of diabetes mellitus, obesity, hypertriglyceridemia, ischemic cerebrovascular disease, ischemic cardiovascular disease, and diabetic nephropathy.

[21] The method according to any one of [15] to [20], wherein the FGF21 expression level is measured by using FGF21 transcription promoting activity as an index.

### Advantageous Effects of Invention

The present invention provides FGF21 as a biomarker that mediates metabolic regulations by Nrf2 activation in vivo.

According to some aspects of the present invention, there is provided use of the biomarker. Specifically, use of the biomarker enables, for example, monitoring a response of a subject administered with an Nrf2 activator; monitoring a response of a subject treated with an Nrf2 activator; predicting a clinical response to a treatment with an Nrf2 activator in a subject; selecting a subject for which a treatment with an Nrf2 activator is effective; and selecting the optimum dose of an Nrf2 activator for a subject in a treatment with an Nrf2 activator.

According to some other aspects of the present invention, there is provided a method for screening for a preventive or therapeutic agent with a novel mechanism of action for Nrf2- and/or FGF21-related disorders, whereby Nrf2 activity is measured by using increase of FGF21 expression level as an index, or a compound that was found to have Nrf2 activation activity is checked for activity that increases FGF21 expression level.

### BRIEF DESCRIPTION OF DRAWINGS

FIG. 1 is a diagram representing Nrf2 activation increasing FGF21 in db/db mice; (a) plasma FGF21 concentration in Keap1 gene knockdown (Keap1^{flox/-}) mutant and littermate control (Keap1^{flox/+}) mice (males and females, n=11 to 17); (b) Fgf21 mRNA expression level in Keap1 gene knockdown (Keap1^{flox/-}) mutant mouse liver (males and females, n=3 to 17); (c) plasma FGF21 concentration after administration of vehicle or Nrf2 activator CDDO-Im (males, n=6 or 7); (d) Fgf21 mRNA expression level in the liver of vehicle- or CDDO-Im-treated mice (males, n=3 to 7). *P<0.05, **P<0.01, ***P<0.001 vs Nrf2^{+/+}::Keap1^{flox/+} mice.

FIG. 2 is a diagram representing CDDO-Im administration inducing FGF21; (a) hepatic Nqo1 expression level and (b) plasma FGF21 level after single administration of CDDO-Im in db/db mice; males, n=3 to 5.

FIG. 3 is a diagram representing Nrf2 increasing FGF21 in diet-induced obesity model mice; (a, b) plasma FGF21 level and (c, d) hepatic Fgf21 mRNA expression level in Keap1^{flox/-} mutant mice. Mice were fed with high calorie diet (HCD) or standard diet (SD) for 8 weeks (males, n=4 to 6). *P<0.05 vs Nrf2^{+/+}::Keap1^{flox/+} mice.

FIG. 4 is a diagram representing Nrf2 decreasing plasma triacylglycerol level and non-esterified fatty acid level; (a) plasma triacylglycerol concentration, (b) plasma non-esterified fatty acid concentration, and (c) blood glucose level in Keap1^{flox/-} mutant mice and littermate Keap1^{flox/+} mice (males and females, n=11 to 13). *P<0.05, ***P<0.001 vs Keap1^{flox/+} mice.

FIG. 5 is a diagram representing CDDO-Im administration and Nrf2 overexpression activating FGF21 promoter, showing reporter activity levels after addition of vehicle or CDDO-Im in Hepa1 cells transfected with Fgf21 reporter and mock, or Fgf21 reporter and Nrf2.

### DESCRIPTION OF EMBODIMENTS

The present invention is described below in detail.

### 1. Nrf2 Activator

Nrf2, or nuclear factor erythroid 2-related factor 2 as it is also called, is a transcription factor belonging to the CNC (Cap ‘n’ collar) transcription factor family. Nrf2 is inactivated by Keap1 in the cytoplasm through ubiqutination and proteasome degradation under unstressed condition. Under stressed condition, Keap1 is modified, and Nrf2 is avoided from proteasome degradation. This causes Nrf2 to translocate into the nucleus from the cytoplasm, and form heterodimer with Maf proteins, upon which Nrf2 binds to the ARE (antioxidant response element) sequence or the EpRE (electrophil response element) sequence, and activates the target genes. The target genes of Nrf2 are members of the gene group having a cytoprotection effect, for example, such as antioxidant genes (e.g., Hmox1, Gcl, and Txrnd1), and detoxication metabolic genes (e.g., Nqo1, Gsts, and Uqt1a6).

The present inventors found that FGF21 is a metabolism-related biomarker that reflects Nrf2 activation in vivo. FGF21 is a key mediator of glucose and lipid metabolism. Nrf2 activation increases FGF21 expression in body parts such as liver. The FGF21 expressed in liver or other tissue is secreted into plasma.

Nrf2 is not particularly limited in the present invention, and may be of, for example, human or mouse origin. The gene and amino acid sequences of human Nrf2 are registered in GenBank with Accession No. NM_006164 (gene) (SEQ ID NO: 1) and Accession No. NP_006155 (protein) (SEQ ID NO: 2), respectively. The gene and amino acid sequences of mouse Nrf2 are registered in GenBank with Accession No. NM_010902 (gene) (SEQ ID NO: 3) and Accession No. NP_035032 (protein) (SEQ ID NO: 4), respectively.

As used herein, Nrf2 is intended to be inclusive of its mutants, provided that these have substantially the same activity. Examples of such mutants include proteins having amino acid sequences with the deletion, substitution, insertion and/or addition of one to several amino acids (for example, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (one to several), 1 to 8, 1 to 7, 1 to 6, 1 to 5, 1 to 4, 1 to 3, 1 to 2, or 1) in the amino acid sequence of Nrf2 above. It is typically preferable to have fewer numbers of amino acid deletions, substitutions, insertions, or additions. The deletion, substitution, insertion, and addition of amino acid residues may occur at the same time in a combination of two or more.

Substantially the same activity includes, for example, activity that controls (enhances or represses) transcription of Nrf2 target genes (specifically, for example, Cpt1b, PGC1α, Nr4a2, and FGF21), and the ARE binding activity. The activity that controls transcription of Nrf2 target genes includes activity that uses changes in the expression level of target genes (specifically, for example, changes in protein level, and changes in mRNA level) as an index, and such activity also constitutes the Nrf2 activity. Changes in the expression level of Nrf2 target genes may be, for example, an increase in the expression level of Cpt1b, an increase in the expression level of PGC1α, a decrease in the expression level of Nr4a2, or an increase in the expression level of FGF21. The transcriptional control of Nrf2 target genes, or changes in the expression level of Nrf2 target genes may be a direct or an indirect effect. By “substantially the same”, it means that activities are the same in nature (for example, physiologically or pharmacologically the same). It is accordingly preferable that the activities, including the ARE binding activity, and the activity that controls transcription of Nrf2 target gene are the same (for example, about 0.01 to 100 times, preferably about 0.1 to 10 times, more preferably 0.5 to 2 times). However, quantitative factors, such as the extent of activity, and the molecular weight of protein may be different.

As used herein, “Nrf2” typically means Nrf2 protein, but may also mean Nrf2 gene, depending on the context.

As used herein, “Nrf2 activator” is a substance that activates Nrf2 activity. “Nrf2 activity” may be, for example, the activity that controls transcription of Nrf2 target genes, or the ARE binding activity of Nrf2, as mentioned above. The substance may be, for example, a low-molecular compound or a high-molecular compound. Other substances, such as siRNA, shRNA, antisense polynucleotides, peptides, proteins, and enzymes also may be used.

As used herein, “low-molecular compound” means an organic or inorganic substance with a molecular weight of 10,000 or less (preferably, a molecular weight of 5,000 or less, more preferably 2,000 or less, particularly preferably 700 or less). As used herein, “high-molecular compound” means an organic substance with a molecular weight above 10,000 (preferably, a molecular weight of 50,000 or more, more preferably 100,000 or more).

“Nrf2 activator” means a substance having activity that activates at least one Nrf2 activity. By “activating” Nrf2 activity, it means up-regulating Nrf2 activity, and this is achievable by, for example, (1) promoting Nrf2 expression, (2) promoting translocation of Nrf2 from cytoplasm to nucleus, (3) promoting binding of Nrf2 to ARE, or (4) inhibiting translocation of Nrf2 from nucleus to cytoplasm.

By “promoting Nrf2 expression”, it means promoting expression of Nrf2 genes, specifically promoting production of Nrf2 protein by promoting any of the series of events that produces protein from Nrf2 gene (including, for example, transcription (mRNA production), and translation (protein production)).

By “promoting translocation of Nrf2 from cytoplasm to nucleus”, it means, for example, promoting translocation into the nucleus through regulation of Nrf2 degradation, or promoting translocation into the nucleus through changes brought in the phosphorylation state of Nrf2. Regulation of Nrf2 activity in the body takes place primarily through degradation. Inhibiting Nrf2 degradation and stabilizing Nrf2 thus represents the most preferable method of Nrf2 activation. S-Alkylation of Keap1 is a known method of inhibiting Nrf2 degradation and promoting translocation of Nrf2 from cytoplasm to nucleus. S-Alkylation of Keap1 at Cys residues changes the Keap1 conformation, and Keap1 becomes saturated with Nrf2 as the Nrf2 remains attached to the Keap1 without being degraded (“Hinge and Latch” model). This allows Nrf2 to accumulate and translocate into the nucleus without having other Nrf2 molecules being captured by Keap1 and degraded. Bardoxolone derivatives (CDDO derivatives) represent a known example of an Nrf2 activator that involves S-alkylation of Keap1 at Cys 151. In a known method of changing the phosphorylation state, the activity of Nrf2 phosphorylating kinases such as GSK-3β and Fyn is regulated to promote translocation of Nrf2 into the nucleus. For example, a GSK-3β inhibitor (SB216763) is known as a compound that promotes translocation of Nrf2 into the nucleus independent of Keap1.

By “promoting binding of Nrf2 to ARE”, it means, for example, promoting formation of an Nrf2 heterodimer with Maf in the nucleus to promote binding of the Nrf2 to the ARE sequence.

By “inhibiting translocation of Nrf2 from nucleus to cytoplasm”, it means, for example, inhibiting Nrf2 from returning to the cytoplasm after it has translocated into the nucleus so that the Nrf2 can keep exhibiting its transcription control activity in the nucleus.

Nrf2 activity may be measured by using known methods, or methods analogous thereto.

For example, in the case of the activity that controls (enhances or represses) transcription, the Nrf2 activity may be measured by observing changes (increase or decrease) in the expression level of Nrf2 target genes. Nrf2 activity also may be measured by isolating a promoter region of the Nrf2 target gene by using an ordinary method, and observing the activity of a reporter gene (for example, luminescence, fluorescence, or chromogenic genes such as luciferase, GFP, and galactosidase) ligated downstream of the promoter region (see, for example, Mol. Cancer Cell Vol. 6 (1) pp. 154-162 (2007)). It is also possible to use Nrf2 protein stabilization (suppressed Nrf2 degradation) as an index of Nrf2 activity. For example, Nrf2 activity may be measured by observing the activity of a reporter gene upon expression of a fusion protein of Nrf2 and reporter gene (see, for example, Genes Cells. Vol. 16(4) pp. 406-415 (2011)).

In the case of the ARE binding activity, the Nrf2 activity may be measured by observing the binding of Nrf2 to ARE with ChIP assay or the like. ChIP assay may be performed by using known methods, or methods analogous thereto (see, for example, Cell Vol. 103 pp 843-852 (2000)). Nrf2 activity also may be measured with an assay system that uses Biacore or ELISA to evaluate the binding of Nrf2-Maf heterodimer to ARE. The ARE binding activity of Nrf2 also may be measured by binding assays using, for example, isothermal titration calorimetry (ITC), dissociation-enhanced lanthanide fluorescence immunoassay (DELFIA), amplified luminescence proximity homogenous assay (ALPHA), sintillation proximity assay (SPA), fluorescence resonance energy transfer (FRET), time-resolved fluorescence resonance energy transfer (TR-FRET), fluorescence polarization (FP), or enzyme fragment complementation (EFC). The ability to form an Nrf2-Maf heterodimer also may be measured as an index of Nrf2 ARE binding activity, and this may be performed by using the methods similar to the ARE binding evaluation methods above.

Examples of the Nrf2 activator include CDD0 derivatives. Examples of CDD0 derivatives include the compounds described in, for example, WO99/65478, WO2004/064723, U.S. Pat. No. 6,326,507, U.S. Pat. No. 6,974,801, and Japanese patent No. 4541548. A CDD0 derivative according to some aspects of the present invention has a structure represented by the following formula (I) described in WO99/65478.

### [Chem. 1]

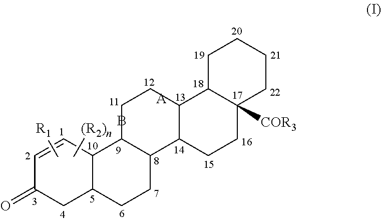

(In the formula,

A or B is a double bond, such that when A is a double bond, C11 has substituted thereon=X which is an organic or inorganic moiety and when B is a double bond, C12 has substituted thereon=X which is an organic or inorganic moiety,

R₁ is an organic or inorganic moiety which is substituted anywhere on the six-membered ring denoted by positions 1 through 10,

R₂ and R₃ are hydrogen or organic or inorganic moieties (an R₂ group is substituted anywhere on the structure represented in formula (I)), and

n is a number from 0 to 100).

The organic moiety and the inorganic moiety are as described from line 15 in page 5 to line 13 in page 7 of WO99/65478. More specifically, the organic moiety includes carbon-based functional groups such as alkyl, alkylamino, alkoxy, aryl, aralkyl, aryloxy, alkylthio, and alkylcarboxyl. The inorganic moiety includes non-carbon-based groups or elements such as hydrogen, halo, amino, nitro, thiol, and hydroxyl. R₁ is, for example, an electron withdrawing group. “Electron withdrawing group” is known in the art, and refers to a group which has a greater electron-withdrawing than hydrogen. A variety of electron-withdrawing groups are known, and include halogens (for example, fluoro, chloro, bromo, and iodo groups), nitro, cyano, —NR₃⁺, —SR₂⁺, —NH₃⁺, —SO₂R, —SO₂Ar, —COOH, —OAr, —COOR, —OR, —COR, —SH, —SR, —OH, —Ar, and —CH═CR₂ (wherein Ar is aryl, and R represents any appropriate organic or inorganic moiety). In a preferred embodiment of formula (I), B is a double bond, and X is O.

A CDD0 derivative according to a more preferred aspect of the present invention is a compound represented by the following formula.

### [Chem. 2]

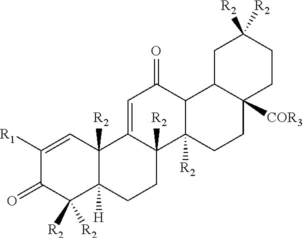

(In the formula,

R₁ is —CN, —COOH, or —COOR (R is alkyl),

R₂ is methyl, and

R₃ is —OH, alkoxy, amino, alkylamino, or —NRₐR_{b} (Rₐ is hydrogen or alkyl, and R_{b} is alkyl or phenyl)).

Here, “alkyl”, “alkoxy”, “amino”, and other groups in the formula are as described from line 29 in page 5 to line 13 in page 7 of WO99/65478. In a more preferred embodiment, the CDD0 derivative is a compound of the foregoing formula in which R₁ is —CN, R₂ is methyl, and R₃ is —OH. In another preferred embodiment, the CDD0 derivative is a compound of the foregoing formula in which R₁ is —CN, R₂ is methyl, and R₃ is —OCH₃.

A CDD0 derivative according to a more preferred aspect of the present invention has a structure represented by the following formula described in WO2004/064723.

### [Chem. 3]

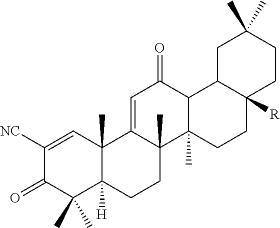

(In the formula, R is a substituted or unsubstituted carbonyl imidazole, CN, CO-D-Glu(OAc)₄, CONH₂, CONHNH₂,

### [Chem. 4]

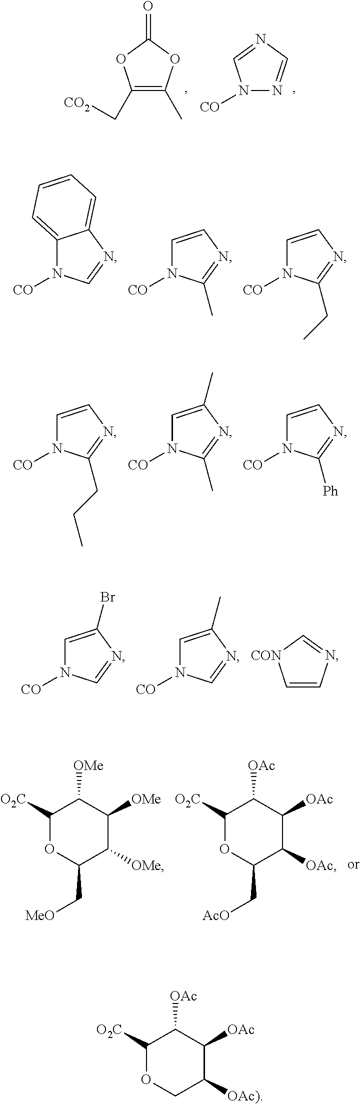

Examples of representative compounds include CDD0-Me (2-cyano-3,12-dioxooleana-1,9(11)-dien-28-oic acid methyl ester), and CDD0-Im (1-[2-cyano-3,12-dioxooleana-1,9(11)-dien-28-oyl]imidazole).

These CDD0 derivatives can be synthesized by using, for example, the method described from line 1 in page 15 to line 15 in page 16 of WO99/65478, the method described from line 15 in page 22 to line 10 in page 24 of WO2004/064723, the method described from line 19 in the right column in page 1027 to line 29 in the right column in page 1028 of T. Honda et al., Bioorganic & medical Chemistry Letters, Vol. 12, pp. 1027-1030 (2002), or methods analogous thereto.

The Nrf2 activator used in the present invention is preferably one having FGF21 level increasing activity. Examples include an Nrf2 activator having activity that increases the expression level (mRNA level) of FGF21 in liver or in hepatocytes, and/or an Nrf2 activator having activity that increases FGF21 protein level in body fluid. The activity that increases FGF21 level can be checked by using, for example, the method described in Example 1-2 or 5-2 below.

The Nrf2 activator used in the present invention preferably has FGF21 transcription promoting activity. For example, an Nrf2 activator may be used that has FGF21 transcription promoting activity in an in vitro promoter assay using hepatocytes. The FGF21 transcription promoting activity can be checked by, for example, a reporter assay using an FGF21 promoter region, such as the method described in Example 6 below.

The Nrf2 activator may be one obtained by using the screening method to be described later.

### 2. Biomarker and Use Thereof

In the present invention, FGF21 is used as a biomarker that mediates metabolic regulations by Nrf2 activation in vivo, as described below.

As used herein, “biomarker” means, for example, an indicator of patient's normal and/or diseased state that can respond to therapeutic intervention. Examples of the biomarker include, but are not limited to, DNA-, RNA-, or protein-based molecular markers. The expression or presence of the biomarker in a biological sample can be detected by standard methods (or the methods disclosed herein), and can be used to, for example, predict a response of a diabetes patient to a treatment with the Nrf2 activator. Such biomarkers considered in the present invention include FGF21, though they are not limited thereto. In some aspects of the invention, the biomarker exists in a test sample in specific amounts or levels different from amounts in a control or reference sample. In another aspect of the invention, expression of such biomarkers may have higher readings than that observed in a control sample. The terms “marker” and “biomarker” as used herein are interchangeable. As used herein, the term “predict” is intended to mean a way of prediction that enables a method for selecting a patient potentially responding to a treatment with the Nrf2 activator.

The term “pharmacodynamic biomarker” means a biomarker that can be detected before, during, and/or after the administration of the Nrf2 activator to a subject in need of administration. The pharmacodynamic biomarker can provide a basis for a clinical or a non-clinical trial assay that helps determine the administration and regimen, identify a subject's subgroup or a phenotype representing a response to a therapeutic agent, or select and develop a major Nrf2 activator. The pharmacological biomarker also can be used to monitor a treatment with the Nrf2 activator.

2.1. Method for Monitoring Response of Subject Administered with Nrf2 Activator, and Method for Monitoring Response of Subject Treated with Nrf2 Activator

According to some aspects of the present invention, there is provided a method for monitoring a response of a subject administered with an Nrf2 activator, or a method for assisting the monitoring (hereinafter, referred to as “administered subject's response monitoring method of the present invention”). The administered subject's response monitoring method of the present invention includes the step of measuring FGF21 level in a biological sample derived from the subject at a time point during or after the administration of the Nrf2 activator. Increase of FGF21 level indicates a positive response to the administration of the Nrf2 activator.

According to some other aspects of the present invention, there is provided a method for monitoring a response of a subject treated with an Nrf2 activator, or a method for assisting the monitoring (hereinafter, referred to as “treated subject's response monitoring method of the present invention”). The treated subject's response monitoring method of the present invention includes the step of measuring FGF21 level in a biological sample derived from the subject at a time point during or after the administration of the Nrf2 activator. Increase of FGF21 level indicates a positive response to the treatment with the Nrf2 activator.

Here, “treatment” includes not only treating and curing Nrf2- and/or FGF21-related disorders, but preventing Nrf2- and/or FGF21-related disorders, or preventing the progression of Nrf2- and/or FGF21-related disorders. The term “response” or “clinical response” is intended to mean changes in the state of a subject in response to administration of the Nrf2 activator or a treatment with the Nrf2 activator, for example, such as changes in the expression level of FGF21 or other variables (specifically, for example, changes in FGF21 protein level, and changes in FGF21 mRNA level), signs of Nrf2- and/or FGF21-related disorders, and alleviation or aggravation of the cause of such signs.

In the present invention, FGF21 is not particularly limited, and may be of, for example, human or mouse origin. The gene and amino acid sequences of human FGF21 are registered in GenBank with Accession No. NM_019113 (gene) (SEQ ID NO: 5) and Accession No. NP_061986 (protein) (SEQ ID NO: 6), respectively. The gene and amino acid sequences of mouse FGF21 are registered in GenBank with Accession No. NM_020013 (gene) (SEQ ID NO: 7) and Accession No. NP_064397 (protein) (SEQ ID NO: 8), respectively.

As used herein, FGF21 is intended to be inclusive of its mutants, provided that these have substantially the same activity. Examples of such mutants include proteins having amino acid sequences with the deletion, substitution, insertion and/or addition of one to several amino acids (for example, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (one to several), 1 to 8, 1 to 7, 1 to 6, 1 to 5, 1 to 4, 1 to 3, 1 to 2, or 1) in the amino acid sequence of Nrf2 above. It is typically preferable to have fewer numbers of amino acid deletions, substitutions, insertions, or additions. The deletion, substitution, insertion, and addition of amino acid residues may occur at the same time in a combination of two or more.

Substantially the same activity includes, for example, the FGF21 receptor binding activity, and the β-Klotho binding activity. By “substantially the same”, it means that activities are the same in nature (for example, physiologically or pharmacologically the same). It is accordingly preferable that the activities, including the FGF21 receptor binding activity, and the β-Klotho binding activity are the same (for example, about 0.01 to 100 times, preferably about 0.1 to 10 times, more preferably 0.5 to 2 times). However, quantitative factors, such as the extent of activity, and the molecular weight of protein may be different. Activities such as the FGF21 receptor binding activity, and the β-Klotho binding activity may be measured according to the methods described in Yie J et al. (2009) FEBS letters, 583, 19-24, and Yie J et al. (2012) Chem Biol Drug Des, 79, 398-410.

As used herein, “FGF21” typically means FGF21 protein, but may also mean FGF21 gene, depending on the context.

“Administration” of the Nrf2 activator means giving the Nrf2 activator to a subject in any route. Specifically, administration may be oral administration or parenteral administration. The administration route may be appropriately selected according to such factors as the type and the dose of the Nrf2 activator, and the dosed subject.

Examples of the Nrf2 activator for oral administration include solid or liquid dosage forms, specifically tablets (including sugar-coated tablets, and film-coated tablets), pills, granules, powders, capsule formulations (including soft capsule formulations), syrups, emulsions, and suspensions. The composition is produced by using methods known per se, and contains a support, a diluent, or an excipient commonly used in the field of pharmaceuticals.

The Nrf2 activator for parenteral administration is, for example, an injection or a suppository. The injection includes dosage forms such as intravenous injection, subcutaneous injection, intradermal injection, intramuscular injection, infusion, and intra-articular injection. The injection is prepared by using methods known per se, for example, by dissolving, suspending, or emulsifying the active component in an aseptic aqueous or oily solution commonly used for injections. A suppository for rectal administration is prepared by mixing the foregoing substances into a common suppository carrier.

The Nrf2 activator may be administered in combination with other drugs or medicinal agents, using a method commonly used in medical practice. Specifically, other drugs or medicinal agents used for combined administration are used for the prevention, delaying, and treatment of Nrf2- and/or FGF21-related disorders, particularly in at least one disorder selected from the group consisting of diabetes mellitus, obesity, hypertriglyceridemia, ischemic cerebrovascular disease, ischemic cardiovascular disease, diabetic nephropathy, and various disorders arising from or associated with these disorders.

For example, insulin resistance improving agents and diabetes drugs may be used. Examples include 1) PPARγ agonists (specifically, for example, pioglitazone, rosiglitazone, troglitazone, ciglitazone, darglitazone, englitazone, and netoglitazone), 2) biguanide drugs (specifically, for example, metformin, buformin, and phenformin), 3) sulfonylurea drugs (specifically, for example, tolbutamide, acetohexamide, chlorpropamide, glibenclamide, gliclazide, glipizide, glimepiride, glipentide, gliquidone, glisolamide, and tolazamide), 4) rapid-acting insulin secretagogues (specifically, for example, nateglinide, mitiglinide, and repaglinide), 5) α-glucosidase inhibitors (specifically, for example, acarbose, voglibose, miglitol, camiglibose, adiposin, emiglitate, pradimicin-Q, and salbostatin), 6) insulins or insulin derivatives (specifically, for example, insulin zinc suspension, insulin lispro, insulin aspart, regular insulin, NPH insulin, insulin glargine, insulin detemir, and mixed insulin), 7) GLP-1 and GLP-1 agonists (specifically, for example, exenatide, liraglutide, lixisenatide, and taspoglutide), 8) DPP-IV inhibitors (specifically, for example, sitagliptin, vildagliptin, alogliptin, saxagliptin, linagliptin, teneligliptin, and NVP-DPP-728), 9) α2 antagonists (specifically, for example, midaglizole, isaglidole, deriglidole, idazoxan, and efaroxan), and 10) SGLT2 inhibitors. Combination drugs containing two or more of these components (specifically, for example, pioglitazone/metformin, and pioglitazone/glimepiride) also may be used.

It is also possible to use hypolipidemic drugs and dyslipidemia treating agents. Examples include 1) ω3 fatty acids (specifically, for example, ethyl icosapentate (EPA-E preparation), and docosahexaenoic acid (DHA)), 2) HMG-CoA reductase inhibitors (specifically, for example, atorvastatin, simvastatin, pitavastatin, itavastatin, fluvastatin, lovastatin, pravastatin, rivastatin, and rosuvastatin), 3) HMG-CoA synthase inhibitors, 4) cholesterol absorption inhibitors (specifically, ezetimibe), 5) acyl-CoA.cholesterol acyltransferase (ACAT) inhibitors, 6) CETP inhibitors, 7) squalene synthase inhibitors, 8) antioxidant (specifically, for example, probucol), 9) PPARα agonists (specifically, for example, clofibrate, etofibrate, fenofibrate, bezafibrate, ciprofibrate, gemfibrozil, and KRP-101), 10) PPARδ agonists, 11) LXRagonists, 12) FXR agonists (specifically, for example, INT-747), 13) MTTP inhibitors, and 14) squalene epoxidase inhibitors, 15) bile acid absorption inhibitors (specifically, for example, colestyramine, and colestipol).

It is also possible to use anti-obesity drugs. Specific examples include 1) CB-1 receptor antagonists (specifically, for example, rimonabant, SR-147778, and BAY-65-2520), 2) monoamine reuptake inhibitors (specifically, for example, sibutramine, and mazindol), 3) serotonin reuptake inhibitors (specifically, for example, fluoxetine, and paroxetine), 4) lipase inhibitors (specifically, for example, orlistat, and cetilistat), 5) neuropeptide Y (NPY) receptor antagonists (specifically, for example, S-2367), 6) peptide YY (PYY) receptor antagonists, 7) adrenaline β3 receptor agonists (specifically, for example, KRP-204, and TRK-380/TAC-301).

When administering the Nrf2 activator in combination with other drugs or medicinal agents, these may be separate preparations or combined preparations. In the case of separate preparations, these may be taken simultaneously, or may be administered at different times.

The language “at a time point during or after the administration of the Nrf2 activator” refers to a time point during continuous (repetitive or intermittent) administration of the Nrf2 activator to a subject, or a time point after the last administration of the Nrf2 activator to a subject. “A time point after the administration” is, for example, after 1 minute to 168 hours (7 days), preferably after 1 hour to 72 hours (3 days), more preferably after 3 hours to 24 hours (1 day), further preferably after 8 hours to 12 hours from the last administration of the Nrf2 activator to a subject.

As used herein, “subject” means an animal to be administered with the Nrf2 activator, and is, for example, a mammal. The mammal may be, for example, a human, a rat, a mouse, a hamster, a rabbit, sheep, a pig, a cow, a cat, a dog, or a monkey. The subject is preferably a human.

In a preferred aspect of the invention, “subject” is a healthy subject, for example, a healthy human. In another preferred aspect, “subject” is a subject affected by Nrf2- and/or FGF21-related disorder. “Nrf2- and/or FGF21-related disorder” means a disorder caused by modulation of Nrf2 and FGF21, specifically down-regulation of Nrf2 and FGF21. More desirably, “subject” is a subject (for example, a human) affected by at least one Nrf2- and/or FGF21-related disorder selected from the group consisting of diabetes mellitus, obesity, hypertriglyceridemia, ischemic cerebrovascular disease, ischemic cardiovascular disease, and diabetic nephropathy. Most preferably, the subject is a subject (for example, a human) affected by diabetes mellitus.

In some aspects of the invention, the subject is a subject that has had continuous administration of the Nrf2 activator or continuous treatment with the Nrf2 activator. The language “has had continuous administration” means that the subject had been administered or treated with the Nrf2 activator before the time of the administration of the Nrf2 activator or the treatment with the Nrf2 activator according to the administered subject's response monitoring method of the present invention, or the treated subject's response monitoring method of the present invention.

“Biological sample” means a sample collected from a subject, and is, for example, any of body fluids, body tissues, cells, tissues, and cell cultures derived from a subject. Specific examples of the biological sample include lymph fluid, whole blood, serum, plasma, urine, saliva, semen, synovial fluid, spinal fluid, and cells or tissues derived from liver. In some aspects of the invention, the biological sample excludes adipose tissues (both brown adipose tissue and white adipose tissue), and cells or tissues derived from pancreas. In a preferred aspect of the invention, the biological sample is a body fluid, more preferably any of whole blood, serum, and plasma. In another preferred aspect, the biological sample is a cell or a tissue derived from liver.

The collection and/or preparation of a biological sample for the measurement of FGF21 level may be performed according to an ordinary method.

“FGF21 level” means FGF21 protein level or FGF21 gene expression level in a biological sample, and is, for example, FGF21 protein level or FGF21 mRNA level. When measuring FGF21 protein level as FGF21 level, the measurement is preferably performed by immunoassay. The immunoassay may be, for example, radioimmunoassay (RIA), fluorescence immunoassay (FIA), luminescence immunoassay, or enzyme immunoassay (for example, enzyme immunoassay (EIA), or enzyme-linked immunosorbent assay (ELISA)). The immunoassay is preferably ELISA. When measuring FGF21 mRNA level as FGF21 level, the measurement may be performed by using known techniques such as PCR.

Increase of FGF21 level is, for example, increase of FGF21 level relative to a reference level. In some aspects of the invention, increase of FGF21 level is increase of FGF21 level relative to an FGF21 level selected from the group consisting of the following (a) to (d):

(a) an FGF21 level in a biological sample derived from the subject at a time point before the administration of the Nrf2 activator;

(b) an FGF21 level in a biological sample derived from a reference population;

(c) a predetermined FGF21 level; and

(d) an FGF21 level in a biological sample derived from the subject at a time point (second time point) before the measurement (first time point) of FGF21 level.

Here, “reference population” includes a subject population before administration of the Nrf2 activator; a subject population under placebo administration; a subject population after placebo administration; a subject population that did not show increase of FGF21 level during administration of the Nrf2 activator; and a subject population that did not show increase of FGF21 level after administration of the Nrf2 activator. Determination of the reference population requires measuring and analyzing FGF21 level in a subject before, during, and/or after administration of the Nrf2 activator. The FGF21 level becomes more reliable as the number of subjects in a population increases. By “before administration of the Nrf2 activator”, it means, for example, a time point before 1 minute to 168 hours (7 days), preferably before 1 hour to 72 hours (3 days), more preferably before 3 hours to 24 hours (1 day), further preferably before 12 hours to 16 hours from the first administration of the Nrf2 activator to a subject. By “during placebo administration”, it means a time period during which the subject is under continuous (repetitive or intermittent) placebo administration. By “after placebo administration”, it means, for example, a time period after 1 minute to 168 hours (7 days), preferably after 1 hour to 72 hours (3 days), more preferably after 3 hours to 24 hours (1 day), further preferably after 8 hours to 12 hours from the last administration of a placebo to a subject. There is a report that FGF21 level shows diurnal variation, and it is preferable to take this into account when setting a time point for measuring the FGF21 level of a reference population. For example, measurement may be made during a time period in which there is small diurnal variation, or during a fixed time period. Specifically, when the time point “after administration of the Nrf2 activator” is 8 hours after the administration, the time point “before administration of the Nrf2 activator” may be 16 hours before the administration.

“Predetermined FGF21 level” can be obtained by measuring the FGF21 level of a subject population before, during, and/or after the administration of the Nrf2 activator and/or a placebo, and analyzing the measurement result. Specifically, the measurement result is statistically analyzed, and the lower limit of FGF21 level that can be determined as a positive response of the subject is determined as the predetermined FGF21 level. Statistical analysis may be performed, for example, by an analysis using a receiver-operating-characteristics (ROC) curve. The reliability of the predetermined FGF21 level can improve as the number of subjects in a population increases. There are reports of FGF21 protein levels for serum biological samples (for example, Galman et al. (2008) Cell Metabolism, 8, 169-174, Xiao et al. (2012) J Clin Endocrinol Metab, 97, E54-E58, Dushay et al. (2010) Gastroenterology, 139, 456-463, Li et al. (2010) Journal of Hepatology, 53, 934-940, and Fisher et al. (2010) Diabetes, 59, 2781-2789), and the predetermined FGF21 protein level also may be set by using these values as a reference.

In a preferred aspect of the invention, a response to administration of the Nrf2 activator or a treatment with the Nrf2 activator can be determined as positive when the FGF21 level is higher than the foregoing FGF21 levels (a) to (d) (hereinafter, referred to as “control”) by a factor of, for example, 1.2 or more, 1.5 or more, 2 or more, 3 or more, 10 or more, 30 or more, or 100 or more.

A “positive response to administration” in the administered subject monitoring method of the present invention includes, for example, at least one selected from the group consisting of positive regulation of FGF21; and improvement of FGF21-related glycolipid metabolism. “Positive regulation of FGF21” means increased FGF21 level, specifically increased FGF21 protein level, or increased FGF21 gene expression level.

A “positive response to treatment” in the treated subject monitoring method of the present invention includes, for example, at least one selected from the group consisting of positive regulation of FGF21; improvement of FGF21-related glycolipid metabolism; and a clinical or therapeutic benefit brought to a subject affected by Nrf2- and/or FGF21-related disorder. “Nrf2- and/or FGF21-related disorder” is as defined above. “Clinical or therapeutic benefit” includes, for example, at least one selected from the group consisting of cellular or biological reaction; complete remission; partial reaction; disease stabilization (no progression or recurrence); and late recurrence.

2.2. Method for Predicting Response (Sensitivity and/or Reactivity) of Subject Treated with Nrf2 Activator

According to some other aspects of the present invention, there is provided a method for predicting a clinical response (sensitivity and/or reactivity) of a subject treated with an Nrf2 activator, or a method for assisting the prediction (hereinafter, “treated subject's response predicting method of the present invention”). The treated subject's response predicting method of the present invention includes the step of measuring FGF21 level in a biological sample derived from the subject at a time point during or after the administration of the Nrf2 activator. Increase of FGF21 level indicates high sensitivity and/or high reactivity to a treatment with the Nrf2 activator.

The Nrf2 activator may be administered orally or parenterally. The administration route may be appropriately selected according to such factors as the type and the dose of the Nrf2 activator, and the dosed subject. The administration, and the Nrf2 activator for oral administration or parenteral administration are as described above.

The Nrf2 activator may be administered in combination with other drugs or medicinal agents. The combined administration is as described above.

“Subject” is as described above, and is, for example, a mammal. The mammal may be, for example, a human, a rat, a mouse, a hamster, a rabbit, sheep, a pig, a cow, a cat, a dog, or a monkey. The subject is preferably a human.

In a preferred aspect of the invention, the subject is a subject affected by Nrf2- and/or FGF21-related disorder. More preferably, “subject” is a subject (for example, a human) affected by at least one disorder selected from the group consisting of diabetes mellitus, obesity, hypertriglyceridemia, ischemic cerebrovascular disease, ischemic cardiovascular disease, and diabetic nephropathy. Most preferably, the subject is a subject (for example, a human) affected by diabetes mellitus.

“Biological sample” is, for example, any of body fluids, body tissues, cells, tissues, and cell cultures derived from a subject, as noted above. Specific examples of the biological sample include lymph fluid, whole blood, serum, plasma, urine, saliva, semen, synovial fluid, spinal fluid, and cells or tissues derived from liver. In some aspects of the invention, the biological sample excludes adipose tissues (both brown adipose tissue and white adipose tissue), and cells or tissues derived from pancreas. In a preferred aspect of the invention, the biological sample is a body fluid, more preferably any of whole blood, serum, and plasma. In another preferred aspect, the biological sample is a cell or a tissue derived from liver.

The collection and/or preparation of a biological sample for the measurement of FGF21 level may be performed according to an ordinary method.

“FGF21 level” is as described above, and is, for example, FGF21 protein level or FGF21 mRNA level. When measuring FGF21 protein level as FGF21 level, the measurement is preferably performed by immunoassay. The immunoassay may be, for example, radioimmunoassay (RIA), fluorescence immunoassay (FIA), luminescence immunoassay, or enzyme immunoassay (for example, enzyme immunoassay (EIA), or enzyme-linked immunosorbent assay (ELISA)). The immunoassay is preferably ELISA. When measuring FGF21 mRNA level as FGF21 level, the measurement may be performed by using known techniques such as PCR.

Increase of FGF21 level is, for example, increase of FGF21 level relative to a reference level. In some aspects of the invention, increase of FGF21 level is increase of FGF21 level relative to an FGF21 level selected from the group consisting of the following (a) to (d):

(a) an FGF21 level in a biological sample derived from the subject at a time point before the administration of the Nrf2 activator;

(b) an FGF21 level in a biological sample derived from a reference population;

(c) a predetermined FGF21 level; and

(d) an FGF21 level in a biological sample derived from the subject at a time point before the measurement of FGF21 level.

“Reference population” and “predetermined FGF21 level” are as described above.

In a preferred aspect of the invention, sensitivity and/or reactivity to a treatment with the Nrf2 activator can be determined as high when the FGF21 level is higher than the foregoing FGF21 levels (a) to (d) (hereinafter, referred to as “control”) by a factor of, for example, 1.2 or more, 1.5 or more, 2 or more, 3 or more, 10 or more, 30 or more, or 100 or more.

“High sensitivity and/or reactivity to treatment” in the treated subject's response predicting method of the present invention includes, for example, at least one selected from the group consisting of positive regulation of FGF21; improvement of FGF21-related glycolipid metabolism; and a clinical or therapeutic benefit brought to a subject affected by Nrf2- and/or FGF21-related disorder. “Positive regulation of FGF21”, “Nrf2- and/or FGF21-related disorder”, and “clinical or therapeutic benefit” are as described above.

2.3. Method for Selecting Subject for which Treatment with Nrf2 Activator is Effective

According to some other aspects of the present invention, there is provided a method for selecting a subject for which a treatment with the Nrf2 activator is effective, or a method for assisting the selection (hereinafter, “subject selection method of the present invention”). The subject selection method of the present invention includes the step of measuring FGF21 level in a biological sample derived from the subject at a time point during or after the administration of the Nrf2 activator. Increase of FGF21 level indicates that the treatment with the Nrf2 activator is effective for the subject.

The Nrf2 activator may be administered orally or parenterally. The administration route may be appropriately selected according to such factors as the type and the dose of the Nrf2 activator, and the dosed subject. The administration, and the Nrf2 activator for oral administration or parenteral administration are as described above.

The Nrf2 activator may be administered in combination with other drugs or medicinal agents. The combined administration is as described above.

“Subject” is as described above, and is, for example, a mammal. The mammal may be, for example, a human, a rat, a mouse, a hamster, a rabbit, sheep, a pig, a cow, a cat, a dog, or a monkey. The subject is preferably a human. In a preferred aspect of the invention, the subject is a subject affected by Nrf2- and/or FGF21-related disorder. More preferably, “subject” is a subject (for example, a human) affected by at least one disorder selected from the group consisting of diabetes mellitus, obesity, hypertriglyceridemia, ischemic cerebrovascular disease, ischemic cardiovascular disease, and diabetic nephropathy. Most preferably, the subject is a subject (for example, a human) affected by diabetes mellitus.

“Biological sample” is, for example, any of body fluids, body tissues, cells, tissues, and cell cultures derived from a subject, as noted above. Specific examples of the biological sample include lymph fluid, whole blood, serum, plasma, urine, saliva, semen, synovial fluid, spinal fluid, and cells or tissues derived from liver. In some aspects of the invention, the biological sample excludes adipose tissues (both brown adipose tissue and white adipose tissue), and cells or tissues derived from pancreas. In a preferred aspect of the invention, the biological sample is a body fluid, more preferably any of whole blood, serum, and plasma. In another preferred aspect, the biological sample is a cell or a tissue derived from liver.

The collection and/or preparation of a biological sample for the measurement of FGF21 level may be performed according to an ordinary method.

“FGF21 level” is as described above, and is, for example, FGF21 protein level or FGF21 mRNA level. When measuring FGF21 protein level as FGF21 level, the measurement is preferably performed by immunoassay. The immunoassay may be, for example, radioimmunoassay (RIA), fluorescence immunoassay (FIA), luminescence immunoassay, or enzyme immunoassay (for example, enzyme immunoassay (EIA), or enzyme-linked immunosorbent assay (ELISA)). The immunoassay is preferably ELISA. When measuring FGF21 mRNA level as FGF21 level, the measurement may be performed by using known techniques such as PCR.

Increase of FGF21 level is, for example, increase of FGF21 level relative to a reference level. In some aspects of the invention, increase of FGF21 level is increase of FGF21 level relative to an FGF21 level selected from the group consisting of the following (a) to (d):

(a) an FGF21 level in a biological sample derived from the subject at a time point before the administration of the Nrf2 activator;

(b) an FGF21 level in a biological sample derived from a reference population;

(c) a predetermined FGF21 level; and

(d) an FGF21 level in a biological sample derived from the subject at a time point before the measurement of FGF21 level.

“Reference population” and “predetermined FGF21 level” are as described above.

In a preferred aspect of the invention, a treatment with the Nrf2 activator can be determined as effective for the subject when the FGF21 level is higher than the foregoing FGF21 levels (a) to (d) (hereinafter, referred to as “control”) by a factor of, for example, 1.2 or more, 1.5 or more, 2 or more, 3 or more, 10 or more, 30 or more, or 100 or more.

By selecting a subject that shows higher FGF21 levels upon administration of the Nrf2 activator, it is possible to screen for a subject for which a treatment with the Nrf2 activator is effective, specifically a subject in which the Nrf2 activator is more likely to show efficacy. When the biological sample is serum, it is desirable that subjects to be administered with the Nrf2 activator have an FGF21 protein level of 10000 pg/mL or less, preferably 3000 pg/mL or less, more preferably 1000 pg/mL or less, further preferably 300 pg/mL or less. From these subjects, a subject that shows an increase of FGF21 level by a factor of, for example, 1.2 or more, 1.5 or more, 2 or more, 3 or more, 10 or more, 30 or more, or 100 or more relative to the level before administration of the Nrf2 activator or the level of a placebo administered group can be screened for as a subject for which a treatment with the Nrf2 activator is more effective. Alternatively, a subject that has a serum FGF21 protein concentration of 300 pg/mL or more, preferably 1000 pg/mL or more, more preferably 3000 pg/mL or more, further preferably 10000 pg/mL or more after the administration of the Nrf2 activator can be screened for as a subject for which a treatment with the Nrf2 activator is more effective.

“Treatment with the Nrf2 activator being effective for the subject” in the subject selection method of the present invention includes, for example, a treatment with the Nrf2 activator bringing a clinical or therapeutic benefit to a subject affected by Nrf2- and/or FGF21-related disorder. “Nrf2- and/or FGF21-related disorder” and “clinical or therapeutic benefit” are as described above.

### 2.4. Method for Selecting Optimum Dose of Nrf2 Activator

According to some other aspects of the present invention, there is provided a method for selecting the optimum dose of an Nrf2 activator for the treatment of a subject with the Nrf2 activator, or a method for assisting the selection (hereinafter, “dose selection method of the present invention”). The dose selection method of the present invention includes the step of measuring FGF21 level in a biological sample derived from the subject at a time point during or after the administration of the Nrf2 activator. The optimum dose of the Nrf2 activator is determined by using increase or decrease of FGF21 level as an index.

The Nrf2 activator may be administered orally or parenterally. The administration route may be appropriately selected according to such factors as the type and the dose of the Nrf2 activator, and the dosed subject. The administration, and the Nrf2 activator for oral administration or parenteral administration are as described above.

The Nrf2 activator may be administered in combination with other drugs or medicinal agents. The combined administration is as described above.

“Subject” is as described above, and is, for example, a mammal. The mammal may be, for example, a human, a rat, a mouse, a hamster, a rabbit, sheep, a pig, a cow, a cat, a dog, or a monkey. The subject is preferably a human. In a preferred aspect of the invention, the subject is a subject affected by Nrf2- and/or FGF21-related disorder. More preferably, “subject” is a subject (for example, a human) affected by at least one disorder selected from the group consisting of diabetes mellitus, obesity, hypertriglyceridemia, ischemic cerebrovascular disease, ischemic cardiovascular disease, and diabetic nephropathy. Most preferably, the subject is a subject (for example, a human) affected by diabetes mellitus.

“Biological sample” is as described above, and is, for example, any of body fluids, body tissues, cells, tissues, and cell cultures derived from a subject. Specific examples of the biological sample include lymph fluid, whole blood, serum, plasma, urine, saliva, semen, synovial fluid, spinal fluid, and cells or tissues derived from liver. In some aspects of the invention, the biological sample excludes adipose tissues (both brown adipose tissue and white adipose tissue), and cells or tissues derived from pancreas. In a preferred aspect of the invention, the biological sample is a body fluid, more preferably any of whole blood, serum, and plasma. In another preferred aspect, the biological sample is a cell or a tissue derived from liver.

The collection and/or preparation of a biological sample for the measurement of FGF21 level may be performed according to an ordinary method.

“FGF21 level” is as described above, and is, for example, FGF21 protein level or FGF21 mRNA level. When measuring FGF21 protein level as FGF21 level, the measurement is preferably performed by immunoassay. The immunoassay may be, for example, radioimmunoassay (RIA), fluorescence immunoassay (FIA), luminescence immunoassay, or enzyme immunoassay (for example, enzyme immunoassay (EIA), or enzyme-linked immunosorbent assay (ELISA)). The immunoassay is preferably ELISA. When measuring FGF21 mRNA level as FGF21 level, the measurement may be performed by using known techniques such as PCR.

Increase or decrease of FGF21 level is, for example, increase or decrease of FGF21 level relative to a reference level. In some aspects of the invention, increase or decrease of FGF21 level is increase or decrease of FGF21 level relative to an FGF21 level selected from the group consisting of the following (a) to (d):

(a) an FGF21 level in a biological sample derived from the subject at a time point before the administration of the Nrf2 activator;

(b) an FGF21 level in a biological sample derived from a reference population;

(c) a predetermined FGF21 level; and

(d) an FGF21 level in a biological sample derived from the subject at a time point before the measurement of FGF21 level.

“Reference population” and “predetermined FGF21 level” are as described above.

Specifically, the optimum dose is determined by using increase or decrease of FGF21 level as an index, as follows. When the biological sample is serum, it is desirable that subjects to be administered with the Nrf2 activator have an FGF21 protein level of 10000 pg/mL or less, preferably 3000 pg/mL or less, more preferably 1000 pg/mL or less, further preferably 300 pg/mL or less. A dose that increases the FGF21 level of such subjects by a factor of, for example, 1.2 or more, 1.5 or more, 2 or more, 3 or more, 10 or more, 30 or more, or 100 or more relative to the level before administration of the Nrf2 activator or the level of a placebo administered group is determined as the optimum dose. Alternatively, a dose that produces a serum FGF21 protein concentration of 300 pg/mL or more, preferably 1000 pg/mL or more, more preferably 3000 pg/mL or more, further preferably 10000 pg/mL or more after the administration of the Nrf2 activator is determined as the optimum dose.

In addition to “increase or decrease of FGF21 level”, the determination of optimum dose may be made taking into account other factors known to healthcare professionals, for which at least one may be selected from the group consisting of, for example, the age, the body weight, and the sex of the subject being treated; clinical conditions of subjects; and the delivery site, the type, the administration method, and the administration schedule of the Nrf2 activator.

The optimum dose so determined represents the minimum dose required to bring a clinical or therapeutic benefit to a subject. Preferably, the optimum dose is lower than amounts that cause serious side effects in the subject.

3. Method for Screening for Preventive or Therapeutic Agent of Nrf2- and/or FGF21-Related Disorder

The present invention provides a method for screening for a preventive or therapeutic agent for Nrf2- and/or FGF21-related disorder using Nrf2 activation activity as an index.

The screening method of the present invention is a method that includes evaluating test compounds for Nrf2 activation activity, and selecting a compound having Nrf2 activation activity. The compound with Nrf2 activation activity selected by the screening method of the present invention becomes a candidate compound for a preventive or therapeutic agent for Nrf2- and/or FGF21-related disorders, preferably at least one selected from the group consisting of diabetes mellitus, obesity, hypertriglyceridemia, ischemic cerebrovascular disease, ischemic cardiovascular disease, and diabetic nephropathy.

Examples of the test compounds include peptides, proteins, antibodies, nonpeptide compounds, synthetic compounds, fermentation products, cell extracts, plant extracts, animal tissue extracts, and plasma. These may be novel compounds or known compounds. The test compounds may form salts, which may be, for example, physiologically acceptable metal salts, ammonium salts, salts with organic bases, salts with inorganic acids, salts with organic acids, or salts with basic or acidic amino acids. Preferred examples of the metal salts include alkali metal salts such as sodium salts, and potassium salts; alkaline-earth metal salts such as calcium salts, magnesium salts, and barium salts; and aluminum salts. Preferred examples of the salts with organic bases include salts with trimethylamine, triethylamine, pyridine, picoline, 2,6-lutidine, ethanolamine, diethanolamine, triethanolamine, cyclohexylamine, dicyclohexylamine, and N,N′-dibenzylethylenediamine. Preferred examples of the salts with inorganic acids include salts with hydrochloric acid, hydrobromic acid, nitric acid, sulfuric acid, and phosphoric acid. Preferred examples of the salts with organic acids include salts with formic acid, acetic acid, trifluoroacetic acid, propionic acid, phthalic acid, fumaric acid, oxalic acid, tartaric acid, maleic acid, citric acid, succinic acid, malic acid, methanesulfonic acid, benzoic acid, benzenesulfonic acid, and p-toluenesulfonic acid. Preferred examples of the salts with basic amino acids include salts with arginine, lysine, and ornithine. Preferred examples of the salts with acidic amino acids include salts with aspartic acid, and glutamic acid.

More specifically, for example, the invention provide a method for screening for a preventive or therapeutic agent for Nrf2- and/or FGF21-related disorder, the method including comparing Nrf2 activity in (i) Nrf2 expressing cells contacted to a test compound, and (ii) Nrf2 expressing cells not contacted to the test compound.

In this method, a test compound is first contacted to Nrf2 expressing cells. The cells may be of any origin, including, for example, human- or mouse-derived cells, preferably, human-derived cells. The “Nrf2 expressing cells” used in the screening method of the present invention are, for example, hepatocytes, mouse embryonic fibroblasts (MEF), HEK 293 cells, or 293 T cells. The “Nrf2 expressing cells” used in the screening method of the present invention may be produced by using common genetic engineering techniques.

The method then measures Nrf2 activity. Specifically, for example, the cells (i) and (ii) are cultured, and measured for each Nrf2 activity. The Nrf2 activity measurement uses increase of FGF21 expression level as an index. Preferably, the index used for the Nrf2 activity measurement is increase of FGF21 expression level in hepatocytes. FGF21 expression level may be measured by using known techniques such as PCR, and immunoblotting.

FGF21 expression level also may be measured by using FGF21 transcription promoting activity as an index. Preferably, the index used for the Nrf2 activity measurement is FGF21 transcription promoting activity in hepatocytes. FGF21 transcription promoting activity may be measured by using known techniques such as a reporter assay using an FGF21 promoter region.

Thereafter, a compound is selected that activates the Nrf2 activity relative to the Nrf2 activity in the cells not contacted to the test compound (control). For example, a test compound that increases the Nrf2 activity in (i) relative to the Nrf2 activity in (ii), specifically a test compound that increases the Nrf2 activity 10% or more, 20% or more, 30% or more, 40% or more, or 50% or more may be selected as a compound having Nrf2 activation activity. Whether the effect is specific to Nrf2 can be checked by using and comparing MEFs derived from Nrf2^{-/-} mice and MEFs derived from Nrf2^{+/+} mice.

The compound selected in the manner described above is an Nrf2 activator with FGF21 level increasing activity, and represents a candidate compound for a preventive or therapeutic agent for Nrf2- and/or FGF21-related disorder.

“Nrf2- and/or FGF21-related disorder” is as described above, and is preferably at least one selected from the group consisting of diabetes mellitus, obesity, hypertriglyceridemia, ischemic cerebrovascular disease, ischemic cardiovascular disease, and diabetic nephropathy. The Nrf2- and/or FGF21-related disorder is more preferably diabetes mellitus.

Another preferred aspect of the screening method of the present invention is a method that includes the following steps.

1) comparing Nrf2 activity in (i) Nrf2 expressing cells contacted to a test compound, and (ii) Nrf2 expressing cells not contacted to the test compound,

2) selecting a test compound that increases the Nrf2 activity in (i) relative to the Nrf2 activity in (ii), and

3) checking that the test compound selected in step 2) has activity that increases FGF21 expression level.

Steps 1) and 2) enable screening of Nrf2 activators. The Nrf2 activity measurement method is as described above.

Preferably, in step 1), the Nrf2 activity is measured by using Nrf2 stabilization as an index.

Preferably, in step 2), a compound is selected that activates the Nrf2 activity relative to the Nrf2 activity in cells not contacted to the test compound (control). For example, a test compound that increases the Nrf2 activity in (i) relative to the Nrf2 activity in (ii), specifically a test compound that increases the Nrf2 activity 10% or more, 20% or more, 30% or more, 40% or more, or 50% or more may be selected as a compound having Nrf2 activation activity.

Preferably, step 3) may be adapted to check for activity that increases FGF21 expression level in hepatocytes.

Primers or probes specific to Nrf2 or FGF21 may be appropriately designed by selecting regions of high sequence specificity from Nrf2 or FGF21 base sequences. Such primers or probes are typically configured from 10 to 40, preferably 15 to 30 bases.

The sequence of the FGF21 promoter region used in reporter assay is not particularly limited, as long as it contains the ARE sequence, and has the intrinsic FGF21 promoter activity. For example, the sequence may be of human or mouse origin. The gene sequence of the FGF21 promoter region may be obtained from database such as UCSG Genome Browser, and a region with promoter activity may be appropriately selected. The selected promoter region may be obtained by using known techniques, such as the method described in Example 6 below (see, for example, Cell Metabolism Vol. 5 pp 415-425 (2007)).

In step 3), the selected test compound can be checked for FGF21 expression level increasing activity, for example, by measuring and comparing FGF21 expression level according to the method described above.

In some aspects of the present invention, in step 3), the selected test compound can be checked for FGF21 expression level increasing activity by using the FGF21 transcription promoting activity of the selected test compound as an index. For example, the activity may be checked by measuring and comparing the FGF21 transcription promoting activity using the method described above.

The compound selected in the manner described above is an Nrf2 activator with FGF21 level increasing activity, and represents a candidate compound for a preventive or therapeutic agent for Nrf2- and/or FGF21-related disorder.

The selected candidate compound is administered to laboratory animals (for example, mice, and rats), and checked for preventive or therapeutic effect for Nrf2- and/or FGF21-related disorder. Known test methods may be used to check preventive or therapeutic effect. Preferably, the Nrf2- and/or FGF21-related disorder is at least one selected from the group consisting of diabetes mellitus, obesity, hypertriglyceridemia, ischemic cerebrovascular disease, ischemic cardiovascular disease, and diabetic nephropathy, more preferably diabetes mellitus.

4. Pharmaceutical Composition for Preventing or Treating Nrf2- and/or FGF21-Related Disorder

The present invention provides an Nrf2 activator as an Nrf2- and/or FGF21-related disorder preventive or therapeutic agent having FGF21 level increasing activity. In the present invention, a pharmaceutical composition containing the Nrf2 activator having FGF21 level increasing activity is used as a preventive or therapeutic agent for Nrf2- and/or FGF21-related disorder. In the present invention, the Nrf2 activator having FGF21 level increasing activity is used in the production of a preventive or therapeutic agent for Nrf2- and/or FGF21-related disorder. The present invention also provides an Nrf2- and/or FGF21-related disorder preventing or treating method that includes administering to a subject in need of prevention or treatment of Nrf2- and/or FGF21-related disorder a therapeutically effective amount of the Nrf2 activator having FGF21 level increasing activity.

The Nrf2 activator having FGF21 level increasing activity (hereinafter, “Nrf2 activator of the present invention”) may be obtained by using the screening method described above.

“Nrf2- and/or FGF21-related disorder” is as described above, and is preferably at least one selected from the group consisting of diabetes mellitus, obesity, hypertriglyceridemia, ischemic cerebrovascular disease, ischemic cardiovascular disease, and diabetic nephropathy, more preferably diabetes mellitus.

In the present invention, the Nrf2 activator of the present invention may be formulated using ordinary means, and used as a pharmaceutical composition for the prevention or treatment of Nrf2- and/or FGF21-related disorder. Alternatively, in the present invention, the Nrf2 activator of the present invention may be formulated using ordinary means, and used for a method for preventing or treating Nrf2- and/or FGF21-related disorder.

Examples of the composition for oral administration include solid or liquid dosage forms, specifically tablets (including sugar-coated tablets, and film-coated tablets), pills, granules, powders, capsule formulations (including soft capsule formulations), syrups, emulsions, and suspensions. The composition is produced by using methods known per se, and contains a support, a diluent, or an excipient commonly used in the field of pharmaceuticals. Examples of supports and excipients for tablet use include lactose, starch, sucrose, and magnesium stearate.

The composition for parenteral administration is, for example, an injection or a suppository. The injection includes dosage forms such as intravenous injection, subcutaneous injection, intradermal injection, intramuscular injection, infusion, and intra-articular injection. The injection is prepared by using methods known per se, for example, by dissolving, suspending, or emulsifying the active component in an aseptic aqueous or oily solution commonly used for injections. Examples of the aqueous solution for injections include physiological saline, and isotonic solution containing glucose or other adjuvants. The aqueous solution may be used with suitable solubilizing agents, for example, such as alcohols (e.g., ethanol), polyalcohols (e.g., propylene glycol, and polyethylene glycol), and non-ionic surfactants [e.g., polysorbate 80, an HCO-50 (polyoxyethylene (50 mol) adduct of hydrogenated castor oil)]. The oily solution may be, for example, sesame oil, or soybean oil, and may be used with solubilizing agents such as benzyl benzoate, and benzyl alcohol. The injection so prepared is typically charged into a suitable ampule. A suppository for rectal administration is prepared by mixing the foregoing substances into a common suppository carrier.

The composition may contain other active components (for example, other diabetes drugs), provided that such other active components do not cause undesirable interactions in the mixture. Preferably, the pharmaceutical composition of the present invention contains the Nrf2 activator of the present invention as the sole active ingredient. The pharmaceutical composition of the present invention may be used with other pharmaceutical compositions (for example, other diabetes drugs).

The dose of the active component (the Nrf2 activator of the present invention) varies with conditions such as its effect, the disorder of interest, the dosed subject, the symptoms, and the administration route. In the case of oral administration, the active component is typically administered, for example, in about 0.1 to 100 mg, preferably about 1.0 to 50 mg, more preferably about 1.0 to 20 mg per day for adults (with a 60-kg body weight). For parenteral administration, it is typically desirable in the case of injection to administer the active component by intravenous injection in about 0.01 to 30 mg, preferably about 0.1 to 20 mg, more preferably about 0.1 to 10 mg per day for adults (with a 60-kg body weight), though the dose varies with conditions such as the disorder of interest, the dosed subject, the symptoms, and the administration route. The active component also may be administered to non-human animals in a converted dose per 60-kg body weight.

It is to be understood that all literature and publications cited in this specification are incorporated herein by reference in their entirety, irrespective of intent.

The objects, features, advantages, and ideas of the present invention will be clearly understood by a skilled person from the descriptions of this specification, and it would be easy for a skilled person to implement the invention from the descriptions of the specification. The embodiments including the best mode for carrying out the invention, and concrete examples of implementation discussed herein serve solely to illustrate or describe the preferred embodiments of the present invention, and do not limit the invention in any ways. It will be understood that various modifications may be made to the invention by a skilled person from the descriptions of the specification, provided such modifications do not exceed the intent and the scope of the invention disclosed herein.

### EXAMPLES

The results of Examples were subjected to the statistical analysis below, unless otherwise stated.

The results are represented as mean±SEM (the standard error of the mean). Statistical significances are evaluated using Student's t-test or ANOVA (analysis of variance) by Bonferroni post hoc test.

### Example 1

### FGF21 Induction by Nrf2 Activation in Diabetic Model Mice (Db/Db Mice)

(1-1) Creation of Mice with Specific Nrf2 Activation

Known Keap1 knockout, knockdown allele (Taguchi et al.,

Mol. Cell. Biol. 30:3016-3026. (2010)), and Nrf2 knockout allele (Itoh et al., Biochem. Biophys. Res. Commun. 236:313-322. (1997)) were used. ICR mice and C57BL/KsJ::db/m mice were purchased from Japan SLC (Shizuoka, Japan) and CLEA Japan (Tokyo, Japan), respectively. The Keap1^{flox/+} and Keap1^{flox/-} mice were obtained by crossing Keap1^{+/-} mice and Keap1^{flox/flox} mice backcrossed with ICR. The db/db::Keap1^{flox/+} mice and db/db::Keap1^{flox/-} mice were obtained by crossing db/+::Keap1^{+/-} mice and db/+::Keap1^{flox/flox} mice backcrossed with ICR. The mice were allowed free access to water and rodent chow, and maintained in a specific pathogen-free environment. All mice were handled according to the regulations of the Standards for Human Care and Use of Laboratory Animals of Tohoku University and Guidelines for Proper Conduct of Animal Experiments of the Ministry of Education, Culture, Sports, Science, and Technology of Japan.

### (1-2) FGF21 Induction by Keap1 Knockdown

Using spontaneous diabetic db/db mice, Nrf2 activated mice (db/db::Keap1^{flox/-}) and control mice (db/db::Keap1^{flox/+}) were compared for plasma FGF21 concentration and hepatic Fgf21 gene expression level.

FGF21 plasma concentration was measured using mouse/rat FGF21 Quantikine ELISA Kit (R & D Systems, Minneapolis, Minn., USA).

Total RNA was extracted from liver tissue using Isogen RNA extract kit (Nippon Gene), and reverse-transcribed to cDNA using SuperScript III (Invitrogen). Quantitative real-time PCR (qPCR) was carried out using TaqMan probes using an ABI 7300 system (Applied Biosystems). Relative expression of genes was normalized against Hprt (hypoxanthine guanine phosphoribosyl transferase) mRNA. The following primers and probes were used:

Fgf21
(forward
(SEQ ID NO: 9)
5′-AGATGGAGCTCTCTATGGATCG-3′,
reverse
(SEQ ID NO: 10)
5′-GGGCTTCAGACTGGTACACAT-3′,
probe
(SEQ ID NO: 11)
5′-FAM-TCAGAGAACTGCTGCTGGAGGAC-TAMRA-3′),
Hprt
(forward
(SEQ ID NO: 12)
5′-CTGGTGAAAAGGACCTCTCG-3′,
reverse
(SEQ ID NO: 13)
5′-TGAAGTACTCATTATAGTCAAGGG-3′,
probe
(SEQ ID NO: 14)
5′-ATCCAACAAAGTCTGGCCTGTATCCAAC-3′)

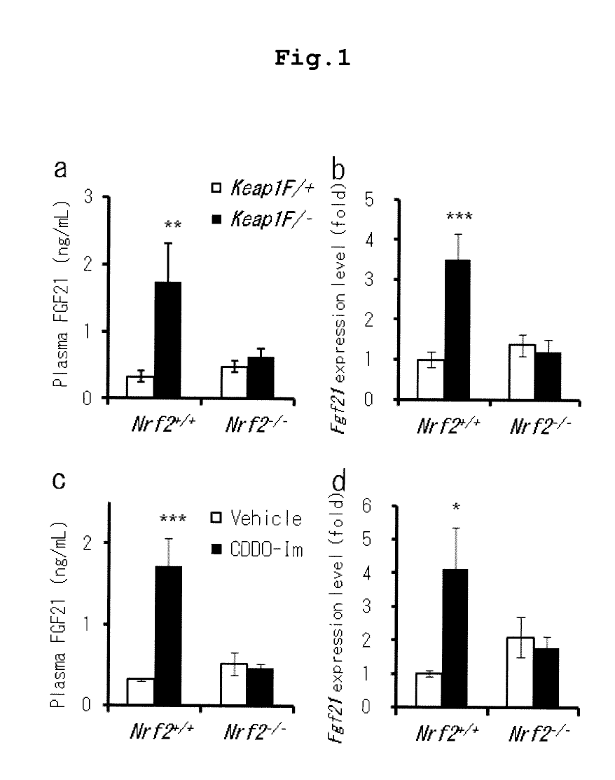

Plasma FGF21 concentration was significantly increased in db/db::Keap1^{flox/-} mice compared with db/db::Keap1^{flox/+} control mice (FIG. 1 (a)). The increase of plasma FGF21 concentration was cancelled by Nrf2 gene knockout (FIG. 1(a)). While hepatic FGF21 mRNA expression level was also significantly increased in db/db::Keap1^{flox/-} mice compared with db/db::Keap1^{flox/+} control mice, there was no difference between db/db::Keap1^{flox/+}::Nrf2^{-/-} mice and db/db::Keap1^{flox/-}::Nrf2^{-/-} mice (FIG. 1(b)).

These results indicate that genetic Nrf2 activation positively regulates hepatic FGF21 gene expression and circulating FGF21 expression level in db/db mice.

### (1-3) FGF21 Induction by Administration of Nrf2 Activator

Using spontaneous diabetic db/db mice, plasma FGF21 concentration and hepatic Fgf21 gene expression level were compared after administration of the Nrf2 activator. CDDO-Im was used as the Nrf2 activator, and orally administered in 10 mg/kg, once a day for 1 week. Blood and tissue were collected under isoflurane anesthesia after certain hours from the administration, and plasma FGF21 concentration and hepatic Fgf21 mRNA expression level were measured in the same manner as in the method described in (1-2).

Oral administration of CDDO-Im to db/db mice significantly increased both plasma FGF21 concentration and hepatic FGF21 gene expression level, and the induction was abolished by Nrf2 gene knockout (FIGS. 1(c) and 1(d)). Taken together, these results show that pharmacological Nrf2 activation also increases hepatic FGF21 gene expression level and plasma FGF21 concentration in diabetic db/db mice as with the case of the Keap1 knockdown mice.

### Example 2

### Time-Course of Nrf2 Activation Marker after Nrf2 Activator Administration

In this Example, time-course of hepatic Nqo1 gene expression and plasma FGF21 concentration were confirmed after Nrf2 activator administration.

CDDO-Im was used as the Nrf2 activator, and orally administered to db/db mice in 30 μmol/kg. Blood and tissue were collected under isoflurane anesthesia after certain hours from the administration, and expression of the Nrf2 target gene Nqo1 in liver and plasma FGF21 concentration were measured in the same manner as in the method described in (1-2). The following primers and probes were used:

Nqo1
(forward
(SEQ ID NO: 15)
5′-AGCTGGAAGCTGCAGACCTG-3′,
reverse
(SEQ ID NO: 16)
5′-CCTTTCAGAATGGCTGGCA-3′,
probe
(SEQ ID NO: 17)
5′-ATTTCAGTTCCCATTGCAGTGGTTTGGG-3′)

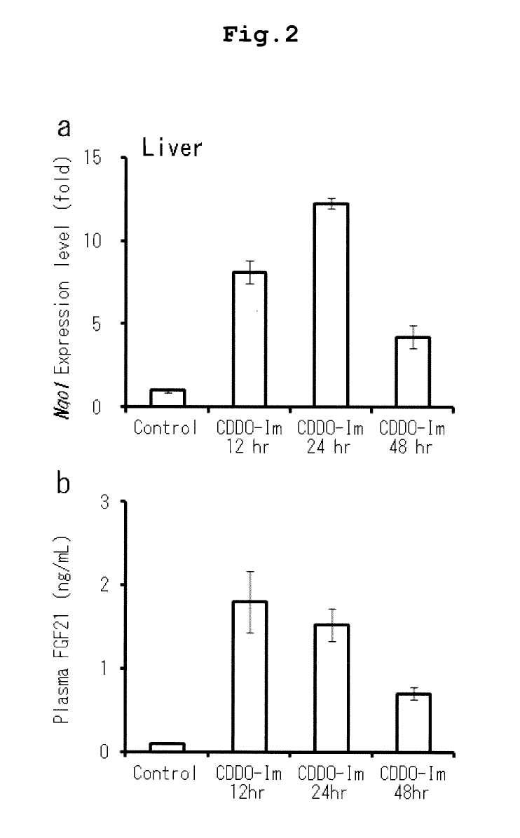

Single administration of CDDO-Im increased NAD(P)H dehydrogenase quinone 1 (Nqo1) gene expression in liver, and the increased expression level was sustained for 48 h after the administration (FIG. 2(a)). Plasma FGF21 concentration was also increased after single administration of CDDO-Im, and was maintained until 48-h postdose, as with the case of the Nqo1 gene expression (FIG. 2(b)). These results indicate that increase of plasma FGF21 concentration closely reflects Nrf2 activation.

### Example 3

### FGF21 Induction by Nrf2 Activation in High-Calorie-Diet-Fed Obesity Model Mice

In this Example, induction of FGF21 by Keap1 knockdown in obesity model mice was confirmed.

In high-calorie-diet (HCD)-fed experiment, standard diet (SD) group was fed with standard chow (MF, 3.59 kcal/g, Oriental Yeast) and HCD group was fed high-fat-diet (HFD-60, 5.06 kcal/g, 62.2% fat of calorie, Oriental Yeast) for 3 weeks followed by concomitant feeding with 20% sucrose in drinking water for 5 weeks.

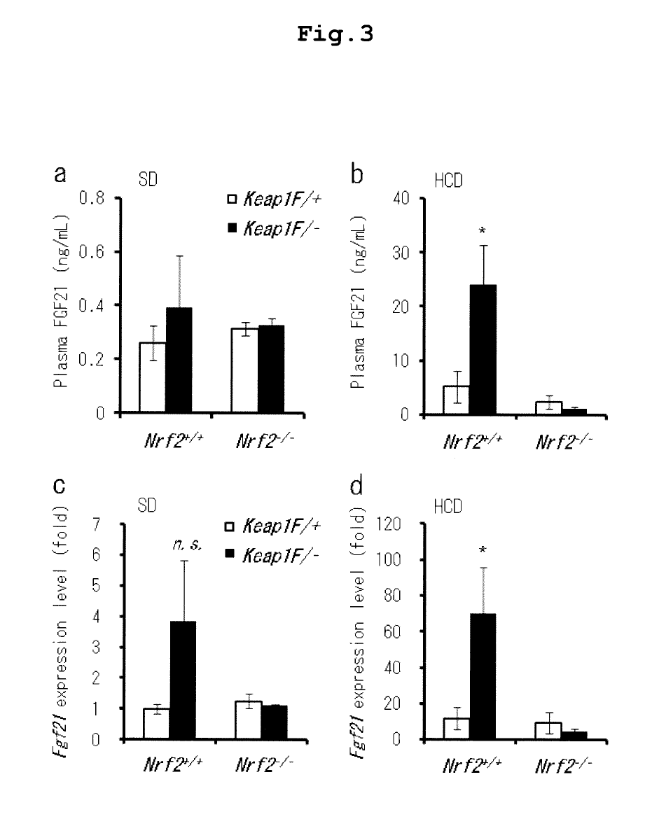

In SD-fed group, plasma FGF21 concentration and hepatic Fgf21 gene expression level tended to increase in Keap1^{flox/-} mice more than in Keap1^{flox/+} control mice (FIGS. 3(a) and 3(c)). In HCD-fed group, both plasma FGF21 concentration and hepatic Fgf21 gene expression level were markedly increased in Keap1^{flox/-} mice compared with Keap1^{flox/+} control mice, and the increase was cancelled by Nrf2 deletion (FIGS. 3(b) and 3(d)).

These results indicate that Nrf2 more strongly induces FGF21 under metabolically stressed conditions.

### Example 4

### Antidiabetic Effect Via FGF21 Induction

In this Example, improvement of glucose metabolism and lipid metabolism was confirmed in db/db mice.

Plasma triacylglycerol (TAG) level and non-esterified fatty acid level were measured using DRI-CHEM 7000 (Fujifilm) and Free Fatty Acid Quantification Kit (BioVision, San Francisco, Calif., USA). Blood glucose level was measured using One Touch Ultra View Blood Glucose Analyzer (LifeScan, Milpitas, Calif., USA).

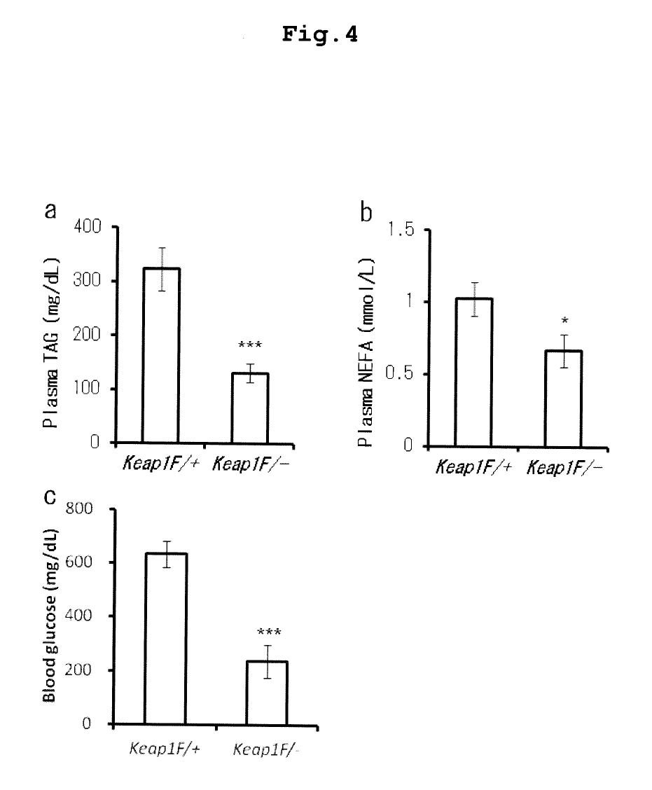

FGF21 is known to improve glucose metabolism and lipid metabolism (Kharitonenkov et al., J. Clin. Invest. 115:1627-1635. (2005)). Plasma triacylglycerol (TAG) level, non-esterified fatty acid (NEFA) level, and blood glucose level were significantly decreased in db/db::Keap1^{flox/-} mice compared with littermate db/db::Keap1^{flox/+} control mice (FIGS. 4(a) to 4(c)).

These results indicate that Nrf2 activation causes marked decrease in plasma lipid level, and that FGF21 can mediate the lipid lowering effect of Nrf2 in diabetic mice.

### Example 5

### Confirmation of Nrf2 Activator Activity

### (5-1) Confirmation of Nrf2 Activation Activity

An Nrf2 activator that stabilizes Nrf2 by inhibiting Nrf2 degradation can be confirmed by using the following method. N0T-MEPs (cells produced from function knockout mice that have Nrf2 replaced its DNA binding domain with LacZ; Hirotsu et al., Genes to Cell, Vol. 16, pp. 406-415 (2011)) are seeded in a 96 well plate, and incubated overnight (35° C., 5% CO₂). The cells are incubated for 3 hours (35° C., 5% CO₂) after adding a test substance. The cells are incubated for 1 hour in the dark at room temperature after adding Beta-Glo Assay Reagent (Promega), and the luminescence intensity is measured to determine Nrf2 activation potency (Nrf2 stabilization potency).

(5-2) Confirmation of Activity that Increases FGF21 Expression Level

Total RNA is extracted from cultured cells or tissues exposed to an Nrf2 activator, using ISOGEN RNA Extract Kit (Nippon Gene), and reverse-transcribed to cDNA using Super-Script III (Invitrogen). FGF21 expression level is confirmed by qPCR performed with FGF21 specific primers. The specific primers may be, for example, the primers used above (Example 1). Gene expression level is normalized with, for example, Hprt. An Nrf2 activator with activity that increases FGF21 expression level relative to the FGF21 expression level in cultured cells or tissues not exposed to the Nrf2 activator is selected.

### Example 6

### Induction of FGF21 Promoter Activation by Nrf2 Activation

In this Example, FGF21 transcription promoting activity by an Nrf2 activator and Nrf2 overexpression was confirmed using FGF21 promoter assay.

Cloning of the mouse FGF21 promoter region was performed using a known technique. By using the mouse Fgf21 cDNA sequence as a template, a sequence (about 2.1 kbp) amplified with the primers 1.1 and 1.2 below was cloned into pGL4.15 vector (Promega) to create a mouse Fgf21 reporter vector.

Primer 1.1:
(SEQ ID NO: 18)
5′-GACGGCCTCGAGGACTGAAGGCTCAGAGACCGG-3′
Primer 1.2:
(SEQ ID NO: 19)
5′-GACGGCAGATCTAGGCAGCTGGAATTGTGTTCTG-3′

The mouse Nrf2 cDNA sequence was cloned into pcDNA3.1/V5-His B vector (Invitrogen) to create a mouse Nrf2 expression vector.

Mouse hepatoma cells Hepa1c1c7 (Hepa1) were cultured in Dulbecco's Modified Eagle medium (Wako) supplemented with 10% fetal bovine serum, and 1% penicillin-streptomycin (Gibco). The mouse Fgf21 reporter vector and mouse Nrf2 expression vector, or the mouse Fgf21 reporter vector and an empty vector (mock) were transfected into Hepa1 cells using Lipofectamine 2000 Transfection Reagent (Invitrogen). The cells were cultured for 6 hours, and the medium was removed. The cells were further cultured for 48 hours after adding a medium containing 100 nM of Nrf2 activator CDDO-Im (2-cyano-3,12-dioxooleana-1,9(11)-dien-28-oic acid imidazol) or 0.1% dimethylsulfoxide (vehicle; Wako). Luciferase activity was then measured using a luminometer (Berthold).

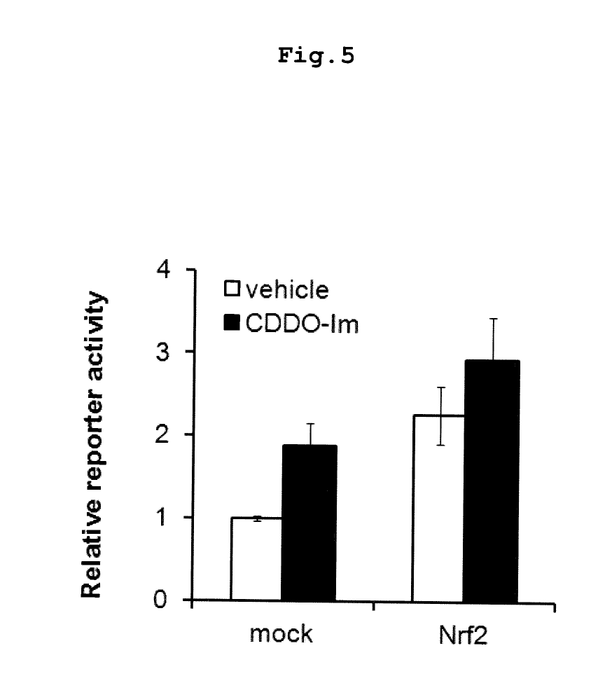

Luciferase activity was enhanced about 2-fold by addition of CDDO-Im compared with the vehicle (FIG. 5). Luciferase activity was also enhanced about 2-fold by transfection of mouse Nrf2 expression vector compared with transfection of empty vector (mock). Luciferase activity was further increased with transfection of mouse Nrf2 expression vector and addition of CDDO-Im.

Nrf2 is a transcription factor that binds to the ARE in the promoters of genes associated with antioxidation and xenobiotic metabolism in response to oxidative and electrophilic stresses to activate their transcription. It was observed in the reporter assay using an upstream sequence of Fgf21 gene that addition of Nrf2 activator CDDO-Im or overexpression of mouse Nrf2 gene increases Fgf21 reporter activity. The upstream sequence of mouse Fgf21 gene contains two ARE sequences, and the results suggest that activation of Nrf2 signaling promotes FGF21 transcription and increases FGF21 expression via the ARE sequences in the Fgf21 promoter region.

### Sequence Listing Free Text

[SEQ ID NO: 1] Base sequence of cDNA encoding human Nrf2 (Accession No. NM_006164)
[SEQ ID NO: 2] Amino acid sequence of human Nrf2 (Accession No. NP_006155)
[SEQ ID NO: 3] Base sequence of cDNA encoding mouse Nrf2 (Accession No. NM_010902)
[SEQ ID NO: 4] Amino acid sequence of mouse Nrf2 (Accession No. NP_035032)
[SEQ ID NO: 5] Base sequence of cDNA encoding human FGF21 (Accession No. NM_019113)
[SEQ ID NO: 6] Amino acid sequence of human FGF21 (Accession No. NP_061986)
[SEQ ID NO: 7] Base sequence of cDNA encoding mouse FGF21 (Accession No. NM_020013)
[SEQ ID NO: 8] Amino acid sequence of mouse FGF21 (Accession No. NP_064397)
[SEQ ID NO: 9 to SEQ ID NO: 19] Primers or probes used in Examples

1. A method for monitoring a response of a subject administered with an Nrf2 activator,
the method comprising the step of measuring FGF21 level in a biological sample derived from the subject at a time point during or after the administration of the Nrf2 activator,
wherein increase of FGF21 level indicates a positive response to the administration of the Nrt2 activator.

2. A method for monitoring a response of a subject treated with an Nrf2 activator,
the method comprising the step of measuring FGF21 level in a biological sample derived from the subject at a time point during or after the administration of the Nrf2 activator,
wherein increase of FGF21 level indicates a positive response to the treatment with the Nrf2 activator.

3.-5. (canceled)

6. The method according to claim 1, wherein the subject is a subject that has had continuous administration of the Nr12 activator.

7. The method according to claim 1, wherein the subject is a healthy subject.

8. The method according to claim 1, wherein the subject is a subject affected by at least one disorder selected from the group consisting of diabetes mellitus, obesity, hypertriglyceridemia, ischemic cerebrovascular disease, ischemic cardiovascular disease, and diabetic nephropathy.

9. The method according to claim 1, wherein the increase of FGF21 level is increase of FGF21 level relative to an FGF21 level selected from the group consisting of the following (a) to (d):
(a) an FGF21 level in a biological sample derived from the subject at a time point before the administration of the Nrf2 activator;
(b) an FGF21 level in a biological sample derived from a reference population;
(c) a predetermined FGF21 level; and
(d) an FGF21 level in a biological sample derived from the subject at a time point before the measurement of FGF21 level.

10. The method according to claim 1, wherein the FGF21 level is FGF21 protein level or FGF21 mRNA level.

11. The method according to claim 10, wherein the FGF21 level is FGF21 protein level.

12. The method according to claim 10, wherein the measurement is performed by immunoassay.

13. The method according to claim 1, wherein the biological sample is a body fluid.

14. The method according to claim 13, wherein the body fluid is selected from the group consisting of whole blood, serum, and plasma.

15.-20. (canceled)

21. The method according to claim 2, wherein the subject is a subject that has had continuous administration of the Nrf2 activator.

22. The method according to claim 2, wherein the subject is a subject affected by at least one disorder selected from the group consisting of diabetes mellitus, obesity, hypertriglyceridemia, ischemic cerebrovascular disease, ischemic cardiovascular disease, and diabetic nephropathy.

23. The method according to claim 2, wherein the increase of FGF21 level is increase of FGF21 level relative to an FGF21 level selected from the group consisting of the following (a) to (d):
(a) an FGF21 level in a biological sample derived from the subject at a time point before the administration of the Nrf2 activator;
(b) an FGF21 level in a biological sample derived from a reference population;
(c) a predetermined FGF21 level; and
(d) an FGF21 level in a biological sample derived from the subject at a time point before the measurement of FGF21 level.

24. The method according to claim 2, wherein the FGF21 level is FGF21 protein level or FGF21 mRNA level.

25. The method according to claim 24, wherein the FGF21 level is FGF21 protein level.

26. The method according to claim 24, wherein the measurement is performed by immunoassay.

27. The method according to claim 2, wherein the biological sample is a body fluid.

28. The method according to claim 27, wherein the body fluid is selected from the group consisting of whole blood, serum, and plasma.


SEQUENCE LISTING (19 sequences)

SEQ ID NO: 1 - DNA, 2884 bp, Homo sapiens
  CDS (556)..(2373)
aaatcagggaggcgcagctcctacaccaacgcctttccggggctccgggtgtgtttgttccaactgtttaaactgtttcaaagcgtccgaactccagcgaccttcgcaaacaactctttatctcgcgggcgagagcgctgcccttatttgcgggggagggcaaactgaacgccggcaccggggagctaacggagacctcctctaggtcccccgcctgctgggaccccagctggcagtcccttcccgcccccggaccgcgagcttcttgcgtcagccccggcgcgggtgggggattttcggaagctcagcccgcgcggccggcgggggaaggaagggcccggactcttgccccgcccttgtggggcgggaggcggagcggggcaggggcccgccggcgtgtagccgattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattaaaaaaaaaaaaaaaaaaaaaaaaaaaaaaaa

SEQ ID NO: 2 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 3 - DNA, 2469 bp, Mus musculus
  CDS (234)..(2027)
tggcccttgcctcttgccctagccttttctccgcctctaagttcttgtcccgtccctaggtccttgttccgcccccagggggcgggggcggggcggactaaggctggcctgccactccagcgagcaggctatctcctagttctccgctgctcggactagccattgccgccgcctcacctctgctgcaagtagcctcgccgtcggggagccctaccacagcgtccgccctcagcatgatggacttggagttgccaccgccaggactacagtcccagcaggacatggatttgattgacatcctttggaggcaagacatagatcttggagtaagtcgagaagtgtttgactttagtcagcgacagaaggactatgagctggaaaaacagaaaaaactcgaaaaggaaagacaagagcaactccagaaggaacaggagaaggccttttttgctcagtttcaactggatgaagaaacaggagaattcctcccaattcagccggcccagcacatccagacagacaccagtggatccgccagctactcccaggttgcccacattcccaaacaagatgccttgtactttgaagactgtatgcagcttttggcagagacattcccatttgtagatgaccatgagtcgcttgccctggatatccccagccacgctgaaagttcagtcttcactgcccctcatcaggcccagtccctcaatagctctctggaggcagccatgactgatttaagcagcatagagcaggacatggagcaagtttggcaggagctattttccattcccgaattacagtgtcttaataccgaaaacaagcagctggctgatactaccgctgttcccagcccagaagccacactgacagaaatggacagcaattaccatttttactcatcgatctcctcgctggaaaaagaagtgggcaactgtggtccacatttccttcatggttttgaggattctttcagcagcatcctctccactgatgatgccagccagctgacctccttagactcaaatcccaccttaaacacagattttggcgatgaattttattctgctttcatagcagagcccagtgacggtggcagcatgccttcctccgctgccatcagtcagtcactctctgaactcctggacgggactattgaaggctgtgacctgtcactgtgtaaagctttcaacccgaagcacgctgaaggcacaatggaattcaatgactctgactctggcatttcactgaacacgagtcccagccgagcgtccccagagcactccgtggagtcttccatttacggagacccaccgcctgggttcagtgactcggaaatggaggagctagatagtgcccctggaagtgtcaaacagaacggccctaaagcacagccagcacattctcctggagacacagtacagcctctgtcaccagctcaagggcacagtgctcctatgcgtgaatcccaatgtgaaaatacaacaaaaaaagaagttcccgtgagtcctggtcatcaaaaagccccattcacaaaagacaaacattcaagccgcttagaggctcatctcacacgagatgagcttagggcaaaagctctccatattccattccctgtcgaaaaaatcattaacctccctgttgatgacttcaatgaaatgatgtccaaggagcaattcaatgaagctcagctcgcattgatccgagatatacgcaggagaggtaagaataaagtcgccgcccagaactgtaggaaaaggaagctggagaacattgtcgagctggagcaagacttgggccacttaaaagacgagagagaaaaactactcagagaaaagggagaaaacgacagaaacctccatctactgaaaaggcggctcagcaccttgtatcttgaagtcttcagcatgttacgtgatgaggatggaaagccttactctcccagtgaatactctctgcagcaaaccagagatggcaatgtgttccttgttcccaaaagcaagaagccagatacaaagaaaaactaggttcgggaggatggagccttttctgagctagtgtttgttttgtactgctaaaacttcctactgtgatgtgaaatgcagaaacactttataagtaactatgcagaattatagccaaagctagtatagcaataatatgaaactttacaaagcattaaagtctcaatgttgaatcagtttcattttaactctcaagttaatttcttaggcaccatttgggagagtttctgtttaagtgtaaatactacagaacttatttatactgttctcacttgttacagtcatagacttatatgacatctggctaaaagcaaactattgaaaactaaccagaccactatacttttttatatactgtatgaacaggaaatgacatttttatattaaattgtttagctcataaaaattaaaaggagctagcactaataaaagaatatcatgactt

SEQ ID NO: 4 - protein, 597 aa, Mus musculus
MMDLELPPPGLQSQQDMDLIDILWRQDIDLGVSREVFDFSQRQKDYELEKQKKLEKERQEQLQKEQEKAFFAQFQLDEETGEFLPIQPAQHIQTDTSGSASYSQVAHIPKQDALYFEDCMQLLAETFPFVDDHESLALDIPSHAESSVFTAPHQAQSLNSSLEAAMTDLSSIEQDMEQVWQELFSIPELQCLNTENKQLADTTAVPSPEATLTEMDSNYHFYSSISSLEKEVGNCGPHFLHGFEDSFSSILSTDDASQLTSLDSNPTLNTDFGDEFYSAFIAEPSDGGSMPSSAAISQSLSELLDGTIEGCDLSLCKAFNPKHAEGTMEFNDSDSGISLNTSPSRASPEHSVESSIYGDPPPGFSDSEMEELDSAPGSVKQNGPKAQPAHSPGDTVQPLSPAQGHSAPMRESQCENTTKKEVPVSPGHQKAPFTKDKHSSRLEAHLTRDELRAKALHIPFPVEKIINLPVDDFNEMMSKEQFNEAQLALIRDIRRRGKNKVAAQNCRKRKLENIVELEQDLGHLKDEREKLLREKGENDRNLHLLKRRLSTLYLEVFSMLRDEDGKPYSPSEYSLQQTRDGNVFLVPKSKKPDTKKN

SEQ ID NO: 5 - DNA, 940 bp, Homo sapiens
  CDS (151)..(780)
ctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggagaaaaaaaaaaaaaaaaaaaaaaaaaaaaaaaaaaaaaaaaaaaaaaaaaaaaaaa

SEQ ID NO: 6 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 7 - DNA, 947 bp, Mus musculus
  CDS (185)..(817)
agacagccttagtgtcttctcagctggggattcaacacaggagaaacagccattcactttgcctgagccccagtctgaacctgacccatccctgctgggcaccggagtcagaacacaattccagctgccttggctcctcagccgctcgcttgccaggggctctcccgaacggagcgcagccctgatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgactcttcctgaatctagggctgtttctttttgggtttccacttatttattacgggtatttatcttatttatttattttagtttttttttcttacttggaataataaagagtctgaaagaaaaatgtgtgtt

SEQ ID NO: 8 - protein, 210 aa, Mus musculus
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 9 - DNA, 22 bp, Artificial sequence
  synthetic DNA
agatggagctctctatggatcg

SEQ ID NO: 10 - DNA, 21 bp, Artificial sequence
  synthetic DNA
gggcttcagactggtacacat

SEQ ID NO: 11 - DNA, 23 bp, Artificial sequence
  synthetic DNA
tcagagaactgctgctggaggac

SEQ ID NO: 12 - DNA, 20 bp, Artificial sequence
  synthetic DNA
ctggtgaaaaggacctctcg

SEQ ID NO: 13 - DNA, 24 bp, Artificial sequence
  Synthetic Construct
tgaagtactcattatagtcaaggg

SEQ ID NO: 14 - DNA, 28 bp, Artificial sequence
  synthetic DNA
atccaacaaagtctggcctgtatccaac

SEQ ID NO: 15 - DNA, 20 bp, Artificial sequence
  synthetic DNA
agctggaagctgcagacctg

SEQ ID NO: 16 - DNA, 19 bp, Artificial sequence
  synthetic DNA
cctttcagaatggctggca

SEQ ID NO: 17 - DNA, 28 bp, Artificial sequence
  synthetic DNA
atttcagttcccattgcagtggtttggg

SEQ ID NO: 18 - DNA, 33 bp, Artificial sequence
  synthetic DNA
gacggcctcgaggactgaaggctcagagaccgg

SEQ ID NO: 19 - DNA, 34 bp, Artificial sequence
  synthetic DNA
gacggcagatctaggcagctggaattgtgttctg